SEQUENCE LISTING (99 sequences)

SEQ ID NO: 1 - RNA, 123 nt, synthetic construct
  misc_feature 1..123: note = Synthetic Heterostructured RNA (NA1001)
  source 1..123: mol_type = other RNA
gggtcgcgcgtttcggtgatgacggtgaaaacctctgacacatgcagctcccggagacggtcacagcttgtctgtaagcggatgccgggagcagacaagcccgccctatagtgagtcgtatta

SEQ ID NO: 2 - RNA, 173 nt, synthetic construct
  misc_feature 1..173: note = Synthetic Heterostructured RNA (NA1501)
  source 1..173: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgccctatagtgagtcgtatta

SEQ ID NO: 3 - RNA, 223 nt, synthetic construct
  misc_feature 1..223: note = Synthetic Heterostructured RNA (NA2001)
  source 1..223: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccctatagtgagtcgtatta

SEQ ID NO: 4 - RNA, 323 nt, synthetic construct
  misc_feature 1..323: note = Synthetic Heterostructured RNA (NA3001)
  source 1..323: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctatccctatagtgagtcgtatta

SEQ ID NO: 5 - RNA, 423 nt, synthetic construct
  misc_feature 1..423: note = Synthetic Heterostructured RNA (NA4001)
  source 1..423: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaccctatagtgagtcgtatta

SEQ ID NO: 6 - RNA, 523 nt, synthetic construct
  misc_feature 1..523: note = Synthetic Heterostructured RNA (NA5001)
  source 1..523: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttccctatagtgagtcgtatta

SEQ ID NO: 7 - RNA, 623 nt, synthetic construct
  misc_feature 1..623: note = Synthetic Heterostructured RNA (NA6001)
  source 1..623: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcccctatagtgagtcgtatta

SEQ ID NO: 8 - RNA, 723 nt, synthetic construct
  misc_feature 1..723: note = Synthetic Heterostructured RNA (NA7001)
  source 1..723: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccctatagtgagtcgtatta

SEQ ID NO: 9 - RNA, 823 nt, synthetic construct
  misc_feature 1..823: note = Synthetic Heterostructured RNA (NA8001)
  source 1..823: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataccctatagtgagtcgtatta

SEQ ID NO: 10 - RNA, 123 nt, synthetic construct
  misc_feature 1..123: note = Synthetic Heterostructured RNA (NA1002)
  source 1..123: mol_type = other RNA
ggggaattccgacaaaaggtggttggtgaagctgacattggactccattctgcttaattgcctaaccccatctcccttcaatctacctaccataaccattttcccctatagtgagtcgtatta

SEQ ID NO: 11 - RNA, 173 nt, synthetic construct
  misc_feature 1..173: note = Synthetic Heterostructured RNA (NA1502)
  source 1..173: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacccctatagtgagtcgtatta

SEQ ID NO: 12 - RNA, 223 nt, synthetic construct
  misc_feature 1..223: note = Synthetic Heterostructured RNA (NA2002)
  source 1..223: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagatccctatagtgagtcgtatta

SEQ ID NO: 13 - RNA, 323 nt, synthetic construct
  misc_feature 1..323: note = Synthetic Heterostructured RNA (NA3002)
  source 1..323: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggaccctatagtgagtcgtatta

SEQ ID NO: 14 - RNA, 423 nt, synthetic construct
  misc_feature 1..423: note = Synthetic Heterostructured RNA (NA4002)
  source 1..423: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatccctatagtgagtcgtatta

SEQ ID NO: 15 - RNA, 523 nt, synthetic construct
  misc_feature 1..523: note = Synthetic Heterostructured RNA (NA5002)
  source 1..523: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgccctatagtgagtcgtatta

SEQ ID NO: 16 - RNA, 623 nt, synthetic construct
  misc_feature 1..623: note = Synthetic Heterostructured RNA (NA6002)
  source 1..623: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttccctatagtgagtcgtatta

SEQ ID NO: 17 - RNA, 723 nt, synthetic construct
  misc_feature 1..723: note = Synthetic Heterostructured RNA (NA7002)
  source 1..723: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaacccctatagtgagtcgtatta

SEQ ID NO: 18 - RNA, 823 nt, synthetic construct
  misc_feature 1..823: note = Synthetic Heterostructured RNA (NA8002)
  source 1..823: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaactattccttggaaatgcaggaacagcaatgcgtccgttgacagcagcagttactgtagctggaggacattcaagatatgttcttgatggagttcctaggatccctatagtgagtcgtatta

SEQ ID NO: 19 - RNA, 336 nt, synthetic construct
  misc_feature 1..336: note = Synthetic Heterostructured RNA (R1)
  source 1..336: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccctatagtgagtcgtatta

SEQ ID NO: 20 - RNA, 414 nt, synthetic construct
  misc_feature 1..414: note = Synthetic Heterostructured RNA (R2)
  source 1..414: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgaccctatagtgagtcgtatta

SEQ ID NO: 21 - RNA, 483 nt, synthetic construct
  misc_feature 1..483: note = Synthetic Heterostructured RNA (R3)
  source 1..483: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctaccctatagtgagtcgtatta

SEQ ID NO: 22 - RNA, 605 nt, synthetic construct
  misc_feature 1..605: note = Synthetic Heterostructured RNA (R4)
  source 1..605: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccctatagtgagtcgtatta

SEQ ID NO: 23 - RNA, 681 nt, synthetic construct
  misc_feature 1..681: note = Synthetic Heterostructured RNA (R5)
  source 1..681: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttaccctatagtgagtcgtatta

SEQ ID NO: 24 - RNA, 750 nt, synthetic construct
  misc_feature 1..750: note = Synthetic Heterostructured RNA (R6)
  source 1..750: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccaccctatagtgagtcgtatta

SEQ ID NO: 25 - RNA, 839 nt, synthetic construct
  misc_feature 1..839: note = Synthetic Heterostructured RNA (R7)
  source 1..839: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccctatagtgagtcgtatta

SEQ ID NO: 26 - RNA, 902 nt, synthetic construct
  misc_feature 1..902: note = Synthetic Heterostructured RNA (R8)
  source 1..902: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctatagtgagtcgtatta

SEQ ID NO: 27 - RNA, 1049 nt, synthetic construct
  misc_feature 1..1049: note = Synthetic Heterostructured RNA (R9)
  source 1..1049: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccctatagtgagtcgtatta

SEQ ID NO: 28 - RNA, 1170 nt, synthetic construct
  misc_feature 1..1170: note = Synthetic Heterostructured RNA (R10)
  source 1..1170: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccccgctgggtgctatcaactcttctctgccgtaccagaacatccacccggttaccatcggtgaatgcccgaaatacgttcgttctgctaaactgcgtatggttaccggtctgcgtaacatccctatagtgagtcgtatta

SEQ ID NO: 29 - RNA, 1665 nt, synthetic construct
  misc_feature 1..1665: note = Synthetic Heterostructured RNA (R11)
  source 1..1665: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccccgctgggtgctatcaactcttctctgccgtaccagaacatccacccggttaccatcggtgaatgcccgaaatacgttcgttctgctaaactgcgtatggttaccggtctgcgtaacatcccgtctatccagtctcgtggtctgttcggtgctatcgctggtttcatcgaaggtggttggaccggtatgatcgacggttggtacggttaccaccaccagaacgaacagggttctggttacgctgctgaccagaaatctacccagaacgctatcaacggtatcaccaacaaagttaacaccgttatcgaaaaaatgaacatccagttcaccgctgttggtaaagaattcaacaaactggaaaaacgtatggaaaacctgaacaaaaaagttgacgacggtttcctggacatctggacctacaacgctgaactgctggttctgctggaaaacgaacgtaccctggacttccacgactctaacgttaaaaacctgtacgaaaaagttaaatctcagctgaaaaacaacgctaaagaaatcggtaacggttgcttcgaattctaccacaaatgcgacaacgaatgcatggaatctgttcgtaacggtacctacgactaccctatagtgagtcgtatta

SEQ ID NO: 30 - RNA, 482 nt, synthetic construct
  misc_feature 1..482: note = Synthetic Heterostructured RNA (NA,NVT,VP1,VP10)
  source 1..482: mol_type = other RNA
gggcgataatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagatcgccctatagtgagtcgtattagtcgactgcagaggcctgcatgcaagcttggcgtaatcatg

SEQ ID NO: 31 - RNA, 493 nt, synthetic construct
  misc_feature 1..493: note = Synthetic Heterostructured RNA (NVT IV,NVT 4,VP20)
  source 1..493: mol_type = other RNA
gggcgataatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagatcgccctatagtgagtcgtattagtcgactgcagaggcctgcatgcaagctttgttgttgttgttgttgttgttg

SEQ ID NO: 32 - RNA, 123 nt, synthetic construct
  misc_feature 1..123: note = Synthetic Heterostructured RNA (NA1001)
  source 1..123: mol_type = other RNA
gggcgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 33 - RNA, 173 nt, synthetic construct
  misc_feature 1..173: note = Synthetic Heterostructured RNA (NA1501)
  source 1..173: mol_type = other RNA
gggcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 34 - RNA, 223 nt, synthetic construct
  misc_feature 1..223: note = Synthetic Heterostructured RNA (NA2001)
  source 1..223: mol_type = other RNA
gggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 35 - RNA, 323 nt, synthetic construct
  misc_feature 1..323: note = Synthetic Heterostructured RNA (NA3001)
  source 1..323: mol_type = other RNA
gggatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 36 - RNA, 423 nt, synthetic construct
  misc_feature 1..423: note = Synthetic Heterostructured RNA (NA4001)
  source 1..423: mol_type = other RNA
gggttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 37 - RNA, 523 nt, synthetic construct
  misc_feature 1..523: note = Synthetic Heterostructured RNA (NA5001)
  source 1..523: mol_type = other RNA
gggaacaatttcacacaggaaacagctatgaccatgattacgaactcgtcctcggttcccggcgatcctctggagataatcgtccacttgccgtcatgcttctttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 38 - RNA, 623 nt, synthetic construct
  misc_feature 1..623: note = Synthetic Heterostructured RNA (NA6001)
  source 1..623: mol_type = other RNA
ggggagcgcaacgcaattaatgtgagttagctcactcattaggcaccccaggctttacactttatgcttccggctcgtatgttgtgtggaattgtgagcggataacaatttcacacaggaaacagctatgaccatgattacgaactcgtcctcggttcccggcgatcctctggagataatcgtccacttgccgtcatgcttctttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 39 - RNA, 723 nt, synthetic construct
  misc_feature 1..723: note = Synthetic Heterostructured RNA (NA7001)
  source 1..723: mol_type = other RNA
gggagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaactcgtcctcggttcccggcgatcctctggagataatcgtccacttgccgtcatgcttctttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 40 - RNA, 823 nt, synthetic construct
  misc_feature 1..823: note = Synthetic Heterostructured RNA (NA8001)
  source 1..823: mol_type = other RNA
gggtatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaactcgtcctcggttcccggcgatcctctggagataatcgtccacttgccgtcatgcttctttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaccctatagtgagtcgtatta

SEQ ID NO: 41 - RNA, 123 nt, synthetic construct
  misc_feature 1..123: note = Synthetic Heterostructured RNA (NA1002)
  source 1..123: mol_type = other RNA
ggggaaaatggttatggtaggtagattgaagggagatggggttaggcaattaagcagaatggagtccaatgtcagcttcaccaaccaccttttgtcggaattcccctatagtgagtcgtatta

SEQ ID NO: 42 - RNA, 173 nt, synthetic construct
  misc_feature 1..173: note = Synthetic Heterostructured RNA (NA1502)
  source 1..173: mol_type = other RNA
ggggttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 43 - RNA, 223 nt, synthetic construct
  misc_feature 1..223: note = Synthetic Heterostructured RNA (NA2002)
  source 1..223: mol_type = other RNA
gggatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 44 - RNA, 323 nt, synthetic construct
  misc_feature 1..323: note = Synthetic Heterostructured RNA (NA3002)
  source 1..323: mol_type = other RNA
gggtccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 45 - RNA, 423 nt, synthetic construct
  misc_feature 1..423: note = Synthetic Heterostructured RNA (NA4002)
  source 1..423: mol_type = other RNA
gggatgcagaaatcctaaaagatgacttcctcaccaccctcaaaactgaatctttcttaaacacccctaatgattttgctgaaatttgggttagtttctttgatccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 46 - RNA, 523 nt, synthetic construct
  misc_feature 1..523: note = Synthetic Heterostructured RNA (NA5002)
  source 1..523: mol_type = other RNA
gggcgattggaaagggatttcgaaccgggtaatttaacagtaccagatatatctttgatgggttctagcacaatctcatggggtttctctgcagtagccactgatgcagaaatcctaaaagatgacttcctcaccaccctcaaaactgaatctttcttaaacacccctaatgattttgctgaaatttgggttagtttctttgatccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 47 - RNA, 623 nt, synthetic construct
  misc_feature 1..623: note = Synthetic Heterostructured RNA (NA6002)
  source 1..623: mol_type = other RNA
gggaagtgttttcaacgcaccaagcatgtaatgaatgtcgtcactactcagtaaattgtcaacaacagtccttccctcagaaagggcagcaagaaggagaatacgattggaaagggatttcgaaccgggtaatttaacagtaccagatatatctttgatgggttctagcacaatctcatggggtttctctgcagtagccactgatgcagaaatcctaaaagatgacttcctcaccaccctcaaaactgaatctttcttaaacacccctaatgattttgctgaaatttgggttagtttctttgatccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 48 - RNA, 723 nt, synthetic construct
  misc_feature 1..723: note = Synthetic Heterostructured RNA (NA7002)
  source 1..723: mol_type = other RNA
ggggttggatttcttcctcagactttttaccgacaggaaactgcccaccacaaccttccacaattgctcgttggttttcattgtcatcttcaacatgaagtccaagtgttttcaacgcaccaagcatgtaatgaatgtcgtcactactcagtaaattgtcaacaacagtccttccctcagaaagggcagcaagaaggagaatacgattggaaagggatttcgaaccgggtaatttaacagtaccagatatatctttgatgggttctagcacaatctcatggggtttctctgcagtagccactgatgcagaaatcctaaaagatgacttcctcaccaccctcaaaactgaatctttcttaaacacccctaatgattttgctgaaatttgggttagtttctttgatccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 49 - RNA, 823 nt, synthetic construct
  misc_feature 1..823: note = Synthetic Heterostructured RNA (NA8002)
  source 1..823: mol_type = other RNA
gggatcctaggaactccatcaagaacatatcttgaatgtcctccagctacagtaactgctgctgtcaacggacgcattgctgttcctgcatttccaaggaatagttggatttcttcctcagactttttaccgacaggaaactgcccaccacaaccttccacaattgctcgttggttttcattgtcatcttcaacatgaagtccaagtgttttcaacgcaccaagcatgtaatgaatgtcgtcactactcagtaaattgtcaacaacagtccttccctcagaaagggcagcaagaaggagaatacgattggaaagggatttcgaaccgggtaatttaacagtaccagatatatctttgatgggttctagcacaatctcatggggtttctctgcagtagccactgatgcagaaatcctaaaagatgacttcctcaccaccctcaaaactgaatctttcttaaacacccctaatgattttgctgaaatttgggttagtttctttgatccaaagaagagagaatttgaaacaagaggacccttttgtgttttagaaagattggaggaattcagactaagggtctgtatcccttgtgccatgctactaatctgtgccatttcttgtgaaaaacttcagtttttccactctctctctgtgttcttggagttgtttgaagaccaaattgtttttttgagaaaattttgaagaaaatggttatggtaggtagattgaagggagatggggttaggcaattaagcagaatggagtccaatgtcagcttcaccaaccaccttttgtcggaattcccctatagtgagtcgtatta

SEQ ID NO: 50 - RNA, 336 nt, synthetic construct
  misc_feature 1..336: note = Synthetic Heterostructured RNA (R1)
  source 1..336: mol_type = other RNA
gggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 51 - RNA, 414 nt, synthetic construct
  misc_feature 1..414: note = Synthetic Heterostructured RNA (R2)
  source 1..414: mol_type = other RNA
gggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 52 - RNA, 483 nt, synthetic construct
  misc_feature 1..483: note = Synthetic Heterostructured RNA (R3)
  source 1..483: mol_type = other RNA
gggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 53 - RNA, 605 nt, synthetic construct
  misc_feature 1..605: note = Synthetic Heterostructured RNA (R4)
  source 1..605: mol_type = other RNA
gggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 54 - RNA, 681 nt, synthetic construct
  misc_feature 1..681: note = Synthetic Heterostructured RNA (R5)
  source 1..681: mol_type = other RNA
gggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 55 - RNA, 750 nt, synthetic construct
  misc_feature 1..750: note = Synthetic Heterostructured RNA (R6)
  source 1..750: mol_type = other RNA
gggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 56 - RNA, 839 nt, synthetic construct
  misc_feature 1..839: note = Synthetic Heterostructured RNA (R7)
  source 1..839: mol_type = other RNA
gggtgaaacgacggttgtagttagaggtaacaacagaaacgtaagcgttttcgttctggtacaggttctgctgttctttagagttcggcgggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 57 - RNA, 902 nt, synthetic construct
  misc_feature 1..902: note = Synthetic Heterostructured RNA (R8)
  source 1..902: mol_type = other RNA
gggtccagtagtagttcatacgaccagcctggtcacgaactttcggacgttcagcgatttccggggtgaaacgacggttgtagttagaggtaacaacagaaacgtaagcgttttcgttctggtacaggttctgctgttctttagagttcggcgggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 58 - RNA, 1049 nt, synthetic construct
  misc_feature 1..1049: note = Synthetic Heterostructured RNA (R9)
  source 1..1049: mol_type = other RNA
gggtctggcatttggtgttgcattcgtgcatagaagcgttagaggtgatgataccagaaccgaaaccacgagacagagcgaaagcgtacatcggagcgatcaggttaccgttagcttcgaagatgatggtgtcacccggtttcagcagggtccagtagtagttcatacgaccagcctggtcacgaactttcggacgttcagcgatttccggggtgaaacgacggttgtagttagaggtaacaacagaaacgtaagcgttttcgttctggtacaggttctgctgttctttagagttcggcgggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 59 - RNA, 1170 nt, synthetic construct
  misc_feature 1..1170: note = Synthetic Heterostructured RNA (R10)
  source 1..1170: mol_type = other RNA
gggatgttacgcagaccggtaaccatacgcagtttagcagaacgaacgtatttcgggcattcaccgatggtaaccgggtggatgttctggtacggcagagaagagttgatagcacccagcggggtctggcatttggtgttgcattcgtgcatagaagcgttagaggtgatgataccagaaccgaaaccacgagacagagcgaaagcgtacatcggagcgatcaggttaccgttagcttcgaagatgatggtgtcacccggtttcagcagggtccagtagtagttcatacgaccagcctggtcacgaactttcggacgttcagcgatttccggggtgaaacgacggttgtagttagaggtaacaacagaaacgtaagcgttttcgttctggtacaggttctgctgttctttagagttcggcgggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 60 - RNA, 1665 nt, synthetic construct
  misc_feature 1..1665: note = Synthetic Heterostructured RNA (R11)
  source 1..1665: mol_type = other RNA
gggtagtcgtaggtaccgttacgaacagattccatgcattcgttgtcgcatttgtggtagaattcgaagcaaccgttaccgatttctttagcgttgtttttcagctgagatttaactttttcgtacaggtttttaacgttagagtcgtggaagtccagggtacgttcgttttccagcagaaccagcagttcagcgttgtaggtccagatgtccaggaaaccgtcgtcaacttttttgttcaggttttccatacgtttttccagtttgttgaattctttaccaacagcggtgaactggatgttcattttttcgataacggtgttaactttgttggtgataccgttgatagcgttctgggtagatttctggtcagcagcgtaaccagaaccctgttcgttctggtggtggtaaccgtaccaaccgtcgatcataccggtccaaccaccttcgatgaaaccagcgatagcaccgaacagaccacgagactggatagacgggatgttacgcagaccggtaaccatacgcagtttagcagaacgaacgtatttcgggcattcaccgatggtaaccgggtggatgttctggtacggcagagaagagttgatagcacccagcggggtctggcatttggtgttgcattcgtgcatagaagcgttagaggtgatgataccagaaccgaaaccacgagacagagcgaaagcgtacatcggagcgatcaggttaccgttagcttcgaagatgatggtgtcacccggtttcagcagggtccagtagtagttcatacgaccagcctggtcacgaactttcggacgttcagcgatttccggggtgaaacgacggttgtagttagaggtaacaacagaaacgtaagcgttttcgttctggtacaggttctgctgttctttagagttcggcgggtggtggataccccacagaaccagaacttctttaccttttttgttaacgtaagagtttttcagtttcgggtaagaaccttctttttcggtcagccacagcaggttacggtagaaagaagatttaccttcgtgagagcaagcagcggtaacaccgttggtgttgtggttcggccaagaagattctttcgggaagatttcgaaacgttcgaaagaagaaacagaagacagctgttcacgcagttcttcgtagtcgatgaagtcacccgggtagcagataccgttttcagagttcggggtttcaacgatgtaagaccaagaacgaaccggcagcagcgggtcgcattccgggttacccagcagccaaccagcgatgttgcatttacccagctgcagcggagcgatacctttcagacggcacagtttaccgttgtgagagtcttccagcaggttaacagagtgggtaacggtaacgtttttttccagaacggtgtcaacggtgtcggtagagttgttagcgtggtaaccgatgcagatggtgtcagcgtcagcagcagccagagcgcacagcagaaccagcaggttagctttcatgagctcggctcccccggaaccgccaccggcctttccctctaggtatgccaccacgctgggtttctccagcagtgatgttaccatccgacttaccgttagttttgccttgtttaggcaagcgtgcaggttgtcgaggtccctatagtgagtcgtatta

SEQ ID NO: 61 - RNA, 475 nt, synthetic construct
  misc_feature 1..475: note = Synthetic Heterostructured RNA (NA,NVT,VP1,VP10)
  source 1..475: mol_type = other RNA
gggcgatctcactttcactctcattttagattctgctctaatccccgtagcctgcgccctactaacattatctgccttatcataatcagttaccggtaaataggcgctagccgtatttctccgcataacagtatcatatatataaggcacagtgaaatgtactgatttttgttctcccaagtggaacgttttagtataagttgagtgtgactgacactcatccgtattagtagatgatcgattatactccgctgatataatcactgttccagtgtgaaacgcatttgaaacaaaatcaaaacgtaactctattggccctgaccaaaagttatacaacccagttacatattccattggggttatttgtccttcatagtcctgatcataaaatctcacacctgagtccaaaactgtattatcgccctatagtgagtcgtattaggatccgatatctagatgcattcgcgaggtaccg

SEQ ID NO: 62 - RNA, 499 nt, synthetic construct
  misc_feature 1..499: note = Synthetic Heterostructured RNA (NVT IV,NVT 4,VP20)
  source 1..499: mol_type = other RNA
gggcgatctcactttcactctcattttagattctgctctaatccccgtagcctgcgccctactaacattatctgccttatcataatcagttaccggtaaataggcgctagccgtatttctccgcataacagtatcatatatataaggcacagtgaaatgtactgatttttgttctcccaagtggaacgttttagtataagttgagtgtgactgacactcatccgtattagtagatgatcgattatactccgctgatataatcactgttccagtgtgaaacgcatttgaaacaaaatcaaaacgtaactctattggccctgaccaaaagttatacaacccagttacatattccattggggttatttgtccttcatagtcctgatcataaaatctcacacctgagtccaaaactgtattatcgccctatagtgagtcgtattaggatccgatatctagatgcattcgcgaggtaccgttgttgttgttgttgttgttgttg

SEQ ID NO: 63 - RNA, 106 nt, synthetic construct
  misc_feature 1..106: note = Synthetic Heterostructured RNA (NA1001)
  source 1..106: mol_type = other RNA
gggtcgcgcgtttcggtgatgacggtgaaaacctctgacacatgcagctcccggagacggtcacagcttgtctgtaagcggatgccgggagcagacaagcccgccc

SEQ ID NO: 64 - RNA, 156 nt, synthetic construct
  misc_feature 1..156: note = Synthetic Heterostructured RNA (NA1501)
  source 1..156: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgccc

SEQ ID NO: 65 - RNA, 206 nt, synthetic construct
  misc_feature 1..206: note = Synthetic Heterostructured RNA (NA2001)
  source 1..206: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccc

SEQ ID NO: 66 - RNA, 306 nt, synthetic construct
  misc_feature 1..306: note = Synthetic Heterostructured RNA (NA3001)
  source 1..306: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctatccc

SEQ ID NO: 67 - RNA, 406 nt, synthetic construct
  misc_feature 1..406: note = Synthetic Heterostructured RNA (NA4001)
  source 1..406: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaccc

SEQ ID NO: 68 - RNA, 506 nt, synthetic construct
  misc_feature 1..506: note = Synthetic Heterostructured RNA (NA5001)
  source 1..506: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttccc

SEQ ID NO: 69 - RNA, 606 nt, synthetic construct
  misc_feature 1..606: note = Synthetic Heterostructured RNA (NA6001)
  source 1..606: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcccc

SEQ ID NO: 70 - RNA, 706 nt, synthetic construct
  misc_feature 1..706: note = Synthetic Heterostructured RNA (NA7001)
  source 1..706: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgctccc

SEQ ID NO: 71 - RNA, 806 nt, synthetic construct
  misc_feature 1..806: note = Synthetic Heterostructured RNA (NA8001)
  source 1..806: mol_type = other RNA
gggtcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataccc

SEQ ID NO: 72 - RNA, 106 nt, synthetic construct
  misc_feature 1..106: note = Synthetic Heterostructured RNA (NA1002)
  source 1..106: mol_type = other RNA
ggggaattccgacaaaaggtggttggtgaagctgacattggactccattctgcttaattgcctaaccccatctcccttcaatctacctaccataaccattttcccc

SEQ ID NO: 73 - RNA, 156 nt, synthetic construct
  misc_feature 1..156: note = Synthetic Heterostructured RNA (NA1502)
  source 1..156: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacccc

SEQ ID NO: 74 - RNA, 206 nt, synthetic construct
  misc_feature 1..206: note = Synthetic Heterostructured RNA (NA2002)
  source 1..206: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagatccc

SEQ ID NO: 75 - RNA, 306 nt, synthetic construct
  misc_feature 1..306: note = Synthetic Heterostructured RNA (NA3002)
  source 1..306: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggaccc

SEQ ID NO: 76 - RNA, 406 nt, synthetic construct
  misc_feature 1..406: note = Synthetic Heterostructured RNA (NA4002)
  source 1..406: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatccc

SEQ ID NO: 77 - RNA, 506 nt, synthetic construct
  misc_feature 1..506: note = Synthetic Heterostructured RNA (NA5002)
  source 1..506: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgccc

SEQ ID NO: 78 - RNA, 606 nt, synthetic construct
  misc_feature 1..606: note = Synthetic Heterostructured RNA (NA6002)
  source 1..606: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttccc

SEQ ID NO: 79 - RNA, 706 nt, synthetic construct
  misc_feature 1..706: note = Synthetic Heterostructured RNA (NA7002)
  source 1..706: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaacccc

SEQ ID NO: 80 - RNA, 806 nt, synthetic construct
  misc_feature 1..806: note = Synthetic Heterostructured RNA (NA8002)
  source 1..806: mol_type = other RNA
ggggaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagaacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaactattccttggaaatgcaggaacagcaatgcgtccgttgacagcagcagttactgtagctggaggacattcaagatatgttcttgatggagttcctaggatccc

SEQ ID NO: 81 - RNA, 319 nt, synthetic construct
  misc_feature 1..319: note = Synthetic Heterostructured RNA (R1)
  source 1..319: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccc

SEQ ID NO: 82 - RNA, 397 nt, synthetic construct
  misc_feature 1..397: note = Synthetic Heterostructured RNA (R2)
  source 1..397: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgaccc

SEQ ID NO: 83 - RNA, 466 nt, synthetic construct
  misc_feature 1..466: note = Synthetic Heterostructured RNA (R3)
  source 1..466: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctaccc

SEQ ID NO: 84 - RNA, 588 nt, synthetic construct
  misc_feature 1..588: note = Synthetic Heterostructured RNA (R4)
  source 1..588: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccc

SEQ ID NO: 85 - RNA, 664 nt, synthetic construct
  misc_feature 1..664: note = Synthetic Heterostructured RNA (R5)
  source 1..664: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttaccc

SEQ ID NO: 86 - RNA, 733 nt, synthetic construct
  misc_feature 1..733: note = Synthetic Heterostructured RNA (R6)
  source 1..733: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccaccc

SEQ ID NO: 87 - RNA, 822 nt, synthetic construct
  misc_feature 1..822: note = Synthetic Heterostructured RNA (R7)
  source 1..822: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccc

SEQ ID NO: 88 - RNA, 885 nt, synthetic construct
  misc_feature 1..885: note = Synthetic Heterostructured RNA (R8)
  source 1..885: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccc

SEQ ID NO: 89 - RNA, 1032 nt, synthetic construct
  misc_feature 1..1032: note = Synthetic Heterostructured RNA (R9)
  source 1..1032: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccc

SEQ ID NO: 90 - RNA, 1153 nt, synthetic construct
  misc_feature 1..1153: note = Synthetic Heterostructured RNA (R10)
  source 1..1153: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccccgctgggtgctatcaactcttctctgccgtaccagaacatccacccggttaccatcggtgaatgcccgaaatacgttcgttctgctaaactgcgtatggttaccggtctgcgtaacatccc

SEQ ID NO: 91 - RNA, 1648 nt, synthetic construct
  misc_feature 1..1648: note = Synthetic Heterostructured RNA (R11)
  source 1..1648: mol_type = other RNA
gggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccccgctgggtgctatcaactcttctctgccgtaccagaacatccacccggttaccatcggtgaatgcccgaaatacgttcgttctgctaaactgcgtatggttaccggtctgcgtaacatcccgtctatccagtctcgtggtctgttcggtgctatcgctggtttcatcgaaggtggttggaccggtatgatcgacggttggtacggttaccaccaccagaacgaacagggttctggttacgctgctgaccagaaatctacccagaacgctatcaacggtatcaccaacaaagttaacaccgttatcgaaaaaatgaacatccagttcaccgctgttggtaaagaattcaacaaactggaaaaacgtatggaaaacctgaacaaaaaagttgacgacggtttcctggacatctggacctacaacgctgaactgctggttctgctggaaaacgaacgtaccctggacttccacgactctaacgttaaaaacctgtacgaaaaagttaaatctcagctgaaaaacaacgctaaagaaatcggtaacggttgcttcgaattctaccacaaatgcgacaacgaatgcatggaatctgttcgtaacggtacctacgactaccc

SEQ ID NO: 92 - RNA, 424 nt, synthetic construct
  misc_feature 1..424: note = Synthetic Heterostructured RNA (NA,NVT,VP1,VP10)
  source 1..424: mol_type = other RNA
gggcgataatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagatcgccc

SEQ ID NO: 93 - RNA, 424 nt, synthetic construct; identical to SEQ ID NO: 92
  misc_feature 1..424: note = Synthetic Heterostructured RNA (NVT IV, NVT 4, VP20)
  source 1..424: mol_type = other RNA

SEQ ID NO: 94 - protein, 2860 aa, unidentified
  REGION 1..2860: note = Chinese Sacbrood virus strain BJ 2012
MDMISPLFYGYVRSTNRFPRSGGIRRDLSAEYSSRARTYKTKLEARNCGVERLSTILTSCKKTFDTVDSYTDLFNGWVSGKYVDKNVHYTEVSSDESGRRIWNVRRAVSIKTAEGIIVWRKVITSYSCRVASELAAKSILVQFVGPVRTQSDEVPSKESIQGDATQQSSKEENTIITRDQQQTVSEKIPSTVGDLAIASSEPTQQFRSLTNRWLPINSIRVTVNSKRNDILAQYYIPEDFLLHYAKCAPNTIPFETYVYGKYELELKFVANGNKFQCGKVIISVKFDSYQADNINTGFQAALSRPHVMLDLSTNNEGVLKIPFRYHRAFVRNQTHKTATVGIRPGKFASIYVQVLSPLQTGEGCANDTFIRPFYRYTRAEFAGTSDKVPLTQTDVIGTLLSGGPTPALKDILVGVERTLDQLGRGNNQDKPKDISSITIIPKPRLGSPHGKGKSDAVAMRVNPVALTSFQDVNAYSDEPRTTLDIARIWGLRSTFNWGSGDAHGKELFNTVLDSGVRFYDQDYEGQITPMEYVTGLYNFWSGPIELRFDFVSNAFHTGTVIISAEYNRSSTNTDECQSHSTYTKTFHLGEQKSVHFTVPYIYDTVMRRNTASAYLPVTDYDKADNVSRAQATGIRAESKMRVKVRVVNVLRPVASTTSTIEVLVYMRGGKNYALHGLKQSTYWPSRSVVPIDSFPPDGYDPVKPTNRPTRGVGSPDSNGGKGQSTVAVSDNPHRFLPANVSNRWNEYSSAYLPRVQMDTGDKEDEDETANFSDGVTAMGFQSLDTQVLIKDILRRPVLLFNHVQLDPGYTGFFIPIMPPSRMMQYKSGDRETSFQRLIGRTPQAAIMNLFRFWRGSLRYTIIIHSTDGHPIYVTHVPHTGNRVYGLMKVNNLHEYTKVPIFGSGLTTEMIIPSVNPSICVEVPFDTENNWAVTFEEDAQRNYSWRDKGDTVTGHLVVTPVVPVYMSVWVEAEDDFEVSNFCGPPTVKTNDWNYAFSDEHARVQMDDSVERVYDEGTQTYYYPAPKPEGFSLNNVRTSVSTLCNMLGKVVTPERAMKTALCATPYFGSAYMTATTLDAIGSMQNTVTGAAHQLTASVDARLEQLSAKFGDSIDVITTAVKEAIGKISSGMFNMVNYTGYCIDVILDILVAWIDRSWTAVGVGIIRFVTKVLGLGAISKVMNMATTFGQLIARVYEPPRPVVQAPPPTEATLTGALAGILGTLMGVYISPLSGGSYFKNLMLRMTSSAGPSYLVGVLRFVEATFNTVKDMILNALGYVSPENAALKMLSGTSATIQNFITDAQLITTEANAALVGHPSFRAKYWNTVMQAYQIQKLLLTVPQSSASPILSRLCSDVIRNSNEKFIDISSSPVRYEPFVICIEGPAGIGKSEIVETLATALLKGVNLKRPHSGATYFRMPGSRFWSGYRDQPVVVYDDWANLTEPQALMQQISELYQLKSTSTFIPEMAHLEEKKIRGNPLIVILLCNHAFPDSAVTNMSLEPSAIYRRRDVLLYAERKPEYEGVSLRDMSVNEQTTFAHLNFYKYKDSTNASSCTSKPVGYELTKEWLVAKFAKWHAQEQIKVQRRMENIRAGMYDAEVGSLRLEDPFSLYYSVSSEVIENNEDVTTGFLPSEILAFECKRIANVIDAHQSSVREIVIPDEPRDPFVTTQGDFAGVFMGAALGRVVLEKVCSFSSELINYAIDWIISKHNVIHECCVCKETKGIAWYCLDSAQLAPQSTHYMCNGCMVASRAANREVVQCPMCRSPNFERWGTYQQMTGITIVGRALIMGLITVDKGVNVLRRMLGGTFGAMYAAIMRIAATLHPSMSERTAELLRMTGALVDMSEYTVRELQHVVTQIDDPFESGDEDDDVGTSKIHWRDIVTFDFEEDVARSLMREREITNIPCLHILLGGALHHVSYRDGGYNVPNGGTMVRVPELPCTSDCYFSDMEAFKSFAQRYKEEKKIEIQSYLLGFINNQHSQDYYRKRVPRVFQPYWMRANEELALEIHNITMDNWYQRVGDSFANYRTLIVAAAGLVMAVGSIFGMYKFFSIGTNPARVEFVPSGDEITRNLKRTTRTLQRTRTERPHFQQVHEHPSLDSVVKKYVARNYITISLYKPAGRVKVNRMWYLWDSGFITEALCAVPIKEAWEKSLKITITPALLEHEEHVYTYDAADFTISESTDLAIWVLSPSFGMFKDIRKFIATDEDLSKPITTEGSLLLAPTNRNPVLKEQSIEILGLQNEMQVSELNGTVFYASDVICYDYSQQGACGSLCFLSRSQRPIVGMHFAGRGEGSCGEGYGVILTKEAIGDILALKSQPVVQLEDWEGPSLEEAKIILPETNVSYIGAVTKEQTPYLHKKTKIRPSLIQNVGDLHPVSEPCILDKTDSRYQHDDTPLVAGCKKHGRLTTDFGTTRVESAKEALWDGWLSKMKPLVVKPKLLTPEEAASGFRDIPYYDPMILNTSAGFPYVATEKKRKEDYIVFERNENEQPIGATIDPSVLEEMKRKSELRRQGVQPITPFIDTLKDERKLPEKVRKYGGTRVFCNPPIDYIVSMRQHYMHFVAAFMEQRFKLMHAVGINVQSTEWTLLASKLLAKGNNICTIDYSNFGPGFNAQIAKAAMELMVRWTMEHVEGVNEIEAHTLLHECLNSVHLVSNTLYQQKCGSPSGAPITVVINTLVNILYIFVAWETQVGSKEKGQTWESFKQNIELFCYGDDLIMSVTDKYKETFNALTISQFLAQYGIVATDANKGEEVEAYTTLLDSTFLKHGFRPHEVYPHLWQSALAWSSINDTTQWIWECADLRLATRENCRAALYQAHGHGSVVYNRFKQQVNKALIKRKIQPIALSWKEIDDLFYPEISY

SEQ ID NO: 95 - DNA, 8192 bp, unidentified
  misc_feature 1..8192: note = Chinese Sacbrood virus strain BJ 2012
  source 1..8192: mol_type = other DNA
atggacatgatttctcctttgttttatggatatgttagaagtaccaaccgattccctaggagtggaggaatcagaagagatttgtctgctgaatatagttcacgcgcccgaacctataagaccaagttggaggcgcgtaattgcggagtggagagattatctacaatccttacctcttgtaagaagacatttgatacagtggactcttataccgatttgtttaatggttgggtttctggtaagtatgttgacaagaatgtccactacaccgaagtgtccagtgatgagagtggacgaagaatctggaatgttagacgtgcagtgtcaattaagaccgcagaaggaattatagtatggcgaaaagttattacctcgtacagttgcagggtggcttcagaattagctgctaagagtatattggtgcaatttgtaggtccagtaaggacccagagtgatgaggtaccctcgaaagaatctattcagggggacgctacacagcaaagttccaaggaagaaaatacaataattacacgagatcagcaacagacagtttcggaaaagataccatcaacagttggggacttggccatagcatcttctgaaccaacgcaacagtttaggagtttaactaatcgttggttgcctattaattcaattagagtaacagtaaatagtaaacggaacgatattttggcacaatattatatcccagaagattttctgttacattatgctaagtgtgcgcctaataccataccatttgagacttatgtgtacggaaaatacgagttagaattgaagttcgtagctaatggtaataagtttcagtgtgggaaagtgattatttctgtaaaatttgacagttatcaagctgataatataaatacagggtttcaagcagccttatcgcgaccgcacgttatgctagatttgtcaactaataacgaaggagttcttaaaataccatttaggtatcaccgtgcctttgttaggaatcaaacgcacaaaacagctacggtgggtataagaccagggaagtttgctagtatttatgtgcaagtgttgtcaccgttacagacgggagagggatgtgcgaacgatacgtttatacgacctttttatcggtatacacgagctgaatttgcgggaacgtcggataaagtcccattgacccagacggatgttataggtactttattatctggagggccaacaccagctcttaaggatattttagtaggtgtagagagaacactggaccagttgggccgaggtaataatcaagataaaccgaaggacataagtagcataactataattccgaaacctcgtttgggttcccctcatgggaaaggtaagagtgatgcagtagccatgcgagtaaatccggtggcactaacatcatttcaagatgtgaacgcttattccgatgagccccgtactacattggatatagctcgtatatggggtttgagaagtacgtttaattggggatcaggagatgcacatggcaaagagttatttaatacagttttggactcaggtgtgagattttatgatcaggactatgaaggacaaataaccccaatggaatatgtaactgggttgtataacttttggtcagggccaatagagttacgttttgattttgtttcaaatgcgtttcacactggaacagtgattatatcagcggagtataatcgatcatctactaatacggatgagtgtcagtcacactcaacttatactaaaacgttccacttgggagaacaaaaatcagtacatttcactgtgccttatatatatgatactgttatgcggagaaatacggctagcgcctatttaccggtaactgattatgataaggcagataatgttagtagggcgcaggctacggggattagagcagaatctaaaatgagagtgaaagtgagagtggttaacgttttaaggcctgtagcctctactacctcaactatagaagttttggtgtatatgcgaggaggaaagaactatgcattgcatggtttaaaacagtcaacgtattggccgtcaaggagtgtggtaccgatagatagttttccacctgatggttatgatccagttaagccaacaaatagaccaacaaggggggtgggttcgccagatagcaatgggggtaagggacaatctacggtagccgtatcagataatccacatagattcctgcccgcgaatgtatctaatcgttggaatgagtattcgagtgcttatttaccgcgggtacaaatggatactggtgataaagaagatgaagatgaaactgctaatttcagtgatggagttacagcgatgggtttccaatctttggatacccaggtattgattaaggatattttgcgaagaccagtgttgttgtttaatcacgtacaattggatcctggctatacaggtttttttatacctataatgccgccttctagaatgatgcaatataaatcaggagatagggaaacatcctttcagcgattgattggtcgtacgccccaagcagcaattatgaatttatttaggttttggagagggtcattgcggtatactattattatacacagtaccgatggtcatcctatatatgtaacgcacgtaccccatacgggaaatagagtgtatggtttaatgaaagtaaataatttacacgagtatacgaaggttcctatttttgggagtggtttaacaactgaaatgattatccctagcgtgaatccgagtatatgtgtagaagtaccatttgatactgaaaataattgggctgtgacatttgaggaagatgctcagcggaattactcatggagagataaaggggatactgtcaccggacacttggttgtaacacctgtagtgccagtttatatgtctgtgtgggtagaagctgaggatgattttgaagtgtctaacttttgtgggccaccgactgtaaagacgaatgattggaattatgcattttctgatgagcatgcgcgagttcagatggatgatagtgtagaaagagtatacgatgagggaacccagacgtattactatccggcccctaagccggagggttttagtttgaacaatgtacggacttcggttagtacgttgtgtaatatgcttgggaaagtagtaactcccgagcgcgcaatgaaaacagcgttgtgcgcaactccctattttgggtcagcttacatgacagctactactctggatgctataggtagtatgcagaataccgttacaggagcagcacatcagctgacggcatcagttgatgcgaggttagagcaactgtcagctaagtttggagattcaatagatgtaatcactacagcggtgaaggaggctataggtaaaatttcttctggtatgtttaatatggttaattatactggttactgtatagacgtgattttggatatattagttgcttggattgaccgaagttggactgcagttggagtaggaattattcgattcgtgactaaggtcttggggttgggtgcaatttctaaagttatgaatatggctacaacttttgggcagcttatagctagggtttatgagcctccacgcccagttgtgcaagcaccacctcctacggaagctacattgactggagctttggcaggtatattgggtacgcttatgggggtttatatatcgccattgtctggaggaagttactttaaaaatttgatgctgaggatgactagttcggctgggccgtcttatttggtgggggtcttacgttttgtagaggcgactttcaatacggtcaaggatatgatattaaatgctttgggttatgtttctccggagaatgcggctctgaaaatgctatcgggaacatcggctacgatacagaattttataactgatgctcagctcattactacagaagctaatgcagcattggtgggacaccctagtttccgtgctaagtattggaacacggtaatgcaagcatatcaaatacagaagttattattgaccgttcctcagtctagtgcctctccaattttatctagattgtgtagtgatgtcatacgtaatagtaatgaaaaatttattgatatctcttcttcaccggtacgttatgaaccttttgtaatttgtattgagggtccagcaggtatcggtaaatcagaaatagtggagaccctagccactgcattattgaaaggagtcaatttaaaacgaccacatagtggggctacgtattttcgaatgccagggtctcggttttggtctggttatagagatcaaccagtggtggtttatgatgattgggctaatttaacggaacctcaagctttaatgcagcaaatctcagaattatatcagctcaaatcaacttctacttttatcccggaaatggctcatttagaagaaaagaagattagaggtaatcccctcattgtgatattattatgtaaccacgcttttccggatagtgcagttactaacatgtctcttgaacccagtgcaatttaccgtaggagagatgtgttattgtatgcagaaagaaaaccagaatatgagggggttagtttaagagacatgagtgttaacgagcaaactacgtttgctcatttgaatttttataagtacaaggattctactaatgcatcctcatgcacatcaaaacctgttggatatgagttgactaaggagtggttggtcgcgaagtttgctaaatggcatgcgcaggaacaaattaaagtacaaagacgtatggagaatatacgggcaggaatgtacgatgctgaggtgggatctttgcgtttggaagatccatttagtttatattactcagtgtctagtgaagttattgaaaataatgaagatgttacgactgggtttcttcctagtgaaattttagcattcgaatgtaagcgtatagcaaacgtgatagatgctcatcaatcctcggtaagggagattgtgattccagatgagccaagagacccatttgttactacacagggtgattttgcgggggtatttatgggagctgctttgggtcgtgtggtactggaaaaagtgtgtagcttttcatcggaactcatcaattatgcgattgattggataattagtaagcataatgtgatccatgagtgttgtgtgtgtaaagagacgaagggtatagcgtggtactgcctggattctgcgcaacttgcccctcaatctacgcattatatgtgcaatggttgtatggtagcgtcgcgtgcagcaaatcgagaagtggtacaatgcccgatgtgtaggagtccaaattttgaacgatggggcacataccaacagatgacaggtataactattgttgggcgcgcattgatcatgggattgataacagttgataagggagttaatgtactccgtcgaatgttgggaggtacctttggcgctatgtatgcggctataatgcgtatagctgctacactccacccttcgatgagtgagagaacagcggagttgttaaggatgacaggggccttggttgatatgagtgaatatactgtgcgagaattgcaacatgtagtaacccagatagatgatccatttgagtcaggggatgaagatgatgatgttggtactagtaaaatacactggcgtgatattgtaacttttgatttcgaggaagatgtggctcgctctctgatgcgagagagggaaattactaatataccctgccttcatatattgttggggggagctttacaccacgtgtcttatcgtgacgggggttataatgtacccaacgggggaactatggtgagagtgccggagttgccgtgtacatcagattgttacttttctgatatggaagcttttaagagttttgcccagcggtataaggaggaaaagaagattgaaattcagtcctatttgttaggttttatcaataatcagcactctcaagactattataggaaacgggttccacgtgtttttcagccttattggatgagagctaatgaagagttagctttagaaattcataatatcactatggataattggtatcagagagtcggtgattcgtttgcgaattacagaactttgatagtggcagccgcaggtttagtgatggcagtgggaagcatatttggaatgtataaattcttttctattggtacgaatccagcgcgtgtagagttcgttcctagtggggatgaaatcactagaaatttgaagcgaacaactagaacattgcaaaggacccgcacggagagacctcattttcaacaagtacatgaacatccatcccttgattctgtggtgaagaagtatgtggctcgtaactatatcaccattagtttgtataagccggcaggacgtgtgaaagttaaccgcatgtggtatttatgggacagtggctttattaccgaggcattatgtgccgtgcctattaaggaggcctgggaaaagagcttaaaaataaccataactccagctctcttggagcatgaggagcatgtctacacttatgatgcagctgatttcactatatctgagtctacggatttggctatttgggtcctgtctccatcatttggaatgtttaaggatataaggaagtttatagctacagatgaggacctatctaaaccaattactacggaagggtccttattattggccccaactaatcgtaacccagtgcttaaggaacagagtatagagatactgggcctacaaaatgaaatgcaagtatctgagttaaatggcacagtattctatgcgagtgatgtaatttgttatgattattcacaacagggagcttgtggatctctgtgcttcttgtcccgctcccaaagacctattgtgggaatgcattttgctggtcgaggtgaggggtcctgtggagaaggttatggggttattttaactaaagaggctattggggatattttagcattgaagtctcaacctgtggtacagttggaagattgggaaggacccagtttagaagaagcaaaaataattttgcctgaaacaaatgtatcttatattggggcggtgacaaaggagcagactccttatctccacaagaaaacaaagataagaccgagtcttattcagaatgtcggtgaccttcatccagtatcagaaccatgtatattggataaaacagattctaggtaccaacatgatgatactcctttggtagcggggtgtaagaaacatgggaggcttaccacagattttggcactacgagagtagaaagcgctaaagaagccctttgggacgggtggctatcgaagatgaaacccttggtggtaaaacccaagttattaacacctgaggaggctgctagtggctttcgtgatataccgtattatgatcctatgattctcaatactagcgcgggttttccttatgtagcaacggaaaagaagcgtaaagaagattatatagtatttgagcgcaatgaaaatgagcaacctattggggcaactatagaccccagcgttctggaagagatgaagcgaaaatctgaattgagaaggcaaggggtgcagcctattacaccatttatcgatacacttaaggatgagaggaaattaccagaaaaagtacgcaagtacggtggaactcgagtattttgtaacccacctattgattatattgtgtcgatgaggcaacactatatgcactttgtggctgcatttatggaacagcgttttaaattaatgcatgctgtggggattaatgtgcaaagtacagaatggaccctcttggcttctaagttgcttgctaaaggaaataatatttgtactattgattattcaaatttcggtccagggtttaatgctcaaatagcaaaagctgctatggaattaatggtgcggtggactatggagcatgttgagggtgtaaatgagatagaagcacacaccttattacatgagtgtttaaattcggttcacttagtatctaatacactgtaccaacagaagtgtggatcacctagtggagcgcccattaccgtagtgataaatactttagttaatattttatatatttttgtagcttgggagacgcaagtaggaagtaaagaaaaagggcaaacttgggaatctttcaaacaaaatattgaattgttttgctacggtgatgatttgataatgtcagttacagataaatataaggaaacttttaatgcgtt

SEQ ID NO: 96 - DNA, 2693 bp, synthetic construct
  misc_feature 1..2693: note = pDM-18T vector
  source 1..2693: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaaagaagcatgacggcaagtggacgattatctccagaggatcgccgggaaccgaggacgagttcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 97 - DNA, 2045 bp, Solanum lycopersicum
  variation 440: note = n is a, c, g, or t
  source 1..2045: mol_type = genomic DNA
gaattccgacaaaaggtggttggtgaagctgacattggactccattctgcttaattgcctaaccccatctcccttcaatctacctaccataaccattttcttcaaaattttctcaaaaaaacaatttggtcttcaaacaactccaagaacacagagagagagtggaaaaactgaagtttttcacaagaaatggcacagattagtagcatggcacaagggatacagacccttagtctgaattcctccaatctttctaaaacacaaaagggtcctcttgtttcaaattctctcttctttggatcaaagaaactaacccaaatttcagcaaaatcattaggggtgtttaagaaagattcagttttgagggtggtgaggaagtcatcttttaggatttctgcatcagtggctactgcagagaaaccccatgagattgtgctagnacccatcaaagatatatctggtactgttaaattacccggttcgaaatccctttccaatcgtattctccttcttgctgccctttctgagggaaggactgttgttgacaatttactgagtagtgacgacattcattacatgcttggtgcgttgaaaacacttggacttcatgttgaagatgacaatgaaaaccaacgagcaattgtggaaggttgtggtgggcagtttcctgtcggtaaaaagtctgaggaagaaatccaactattccttggaaatgcaggaacagcaatgcgtccgttgacagcagcagttactgtagctggaggacattcaagatatgttcttgatggagttcctaggatgagagagagaccaattggtgatttggttgatggtcttaagcagcttggcgcagaggtagattgttcccttggtacgaattgtcccccagttcgaattgtcagcaagggaggtcttccaggagggaaggtaaagctctctggatccatcagcagccaatacctgactgctctgcttatggctgctcccctggctctaggagatgtggagattgaaataattgacaaactgatatctgtgccttatgttgaaatgacactgaagttgatggagcgatttggtgtctttgtggagcacagtagtggctgggacagattcttggtaaaaggaggtcagaagtacaaatctcctgggaaagcatttgttgaaggagatgcctcaagtgctagctattttttggcgggggcagcagtcacaggtggaaccgtcactgttgaaggttgtggaacaagcagtttacagggagatgttaagttcgctgaggtcctcgagaagatgggggcagaagttacatggacagagaacagtgtcacagttaaaggacctccgaggaactcttctggaatgaaacatttgcgtgccattgacgtgaacatgaacaaaatgccagatgtggccatgactcttgccgtagttgcactttttgctgatggtcctactaccataagagacgttgctagctggagagtaaaggaaactgagcggatgattgccatatgcaccgaacttaggaagttgggtgcaacagttgttgaagggtcagactactgcataatcaccccaccagaaaagttaaacgtaacggagattgatacatatgatgaccacagaatggctatggctttctctcttgctgcttgtgctgatgttccagtcactattaagaaccctggctgtactcgcaaaaccttccccgactacttcgaggttctccagaagtactctaagcactaaaccacttcacatgtagaaggaattattttgtactacaagagaaattatgcaccagtttgcaaccaaaatggtgcccataccggaagagaaaaaagctttccaactcctttttatatgtctatgtgagatcatgttcattgtatttgttgaagttgagcttctttttttgtttctcgtgtagaagacatgtatactatatagttaagtacacttccttgaagaatatttaccattgattatcaccgttttagttattgcattttggtattcaaaataaatttgtttccaggatt

SEQ ID NO: 98 - DNA, 2116 bp, synthetic construct
  misc_feature 1..2116: note = Synthetic DNA (Group 3)
  source 1..2116: mol_type = other DNA
taactttaagaaggagatataccatgaaacaccaccaccaccaccaccaccacggtggtctggttccgcgtggttcccatggcggatccccaagcaaactcgcgctaatccaggaacttcccgaccgcattcaaacggcggtggaagcagccatgggaatgagctaccaagacgcaccgaacaacgtgcgcagggacctcgacaacctgcacgcttgcctaaacaaggcaaaactaacggtaagtcggatggtaacatcactgctggagaaacccagcgtggtggcatacctagagggaaaggccggtggcggttccgggggagccgagctcatgaaagctaacctgctggttctgctgtgcgctctggctgctgctgacgctgacaccatctgcatcggttaccacgctaacaactctaccgacaccgttgacaccgttctggaaaaaaacgttaccgttacccactctgttaacctgctggaagactctcacaacggtaaactgtgccgtctgaaaggtatcgctccgctgcagctgggtaaatgcaacatcgctggttggctgctgggtaacccggaatgcgacccgctgctgccggttcgttcttggtcttacatcgttgaaaccccgaactctgaaaacggtatctgctacccgggtgacttcatcgactacgaagaactgcgtgaacagctgtcttctgtttcttctttcgaacgtttcgaaatcttcccgaaagaatcttcttggccgaaccacaacaccaacggtgttaccgctgcttgctctcacgaaggtaaatcttctttctaccgtaacctgctgtggctgaccgaaaaagaaggttcttacccgaaactgaaaaactcttacgttaacaaaaaaggtaaagaagttctggttctgtggggtatccaccacccgccgaactctaaagaacagcagaacctgtaccagaacgaaaacgcttacgtttctgttgttacctctaactacaaccgtcgtttcaccccggaaatcgctgaacgtccgaaagttcgtgaccaggctggtcgtatgaactactactggaccctgctgaaaccgggtgacaccatcatcttcgaagctaacggtaacctgatcgctccgatgtacgctttcgctctgtctcgtggtttcggttctggtatcatcacctctaacgcttctatgcacgaatgcaacaccaaatgccagaccccgctgggtgctatcaactcttctctgccgtaccagaacatccacccggttaccatcggtgaatgcccgaaatacgttcgttctgctaaactgcgtatggttaccggtctgcgtaacatcccgtctatccagtctcgtggtctgttcggtgctatcgctggtttcatcgaaggtggttggaccggtatgatcgacggttggtacggttaccaccaccagaacgaacagggttctggttacgctgctgaccagaaatctacccagaacgctatcaacggtatcaccaacaaagttaacaccgttatcgaaaaaatgaacatccagttcaccgctgttggtaaagaattcaacaaactggaaaaacgtatggaaaacctgaacaaaaaagttgacgacggtttcctggacatctggacctacaacgctgaactgctggttctgctggaaaacgaacgtaccctggacttccacgactctaacgttaaaaacctgtacgaaaaagttaaatctcagctgaaaaacaacgctaaagaaatcggtaacggttgcttcgaattctaccacaaatgcgacaacgaatgcatggaatctgttcgtaacggtacctacgactacccgaaatactctgaagaatctaaactgaaccgtgaaaaagttgacggtgttaaactggaatctatgggtatctaccagatcctggctatctactctaccgttgcttcttctctggttctgctggtttctctgggtgctatctctttctggatgtgctctaacggttctctgcagtgccgtatctgcatctaaggatccgaattcgagctccgtcgacaagcttgcggccgcactcgagcaccaccaccaccaccactgagatccggctgctaacaaag

SEQ ID NO: 99 - DNA, 18 bp, synthetic construct
  misc_feature 1..18: note = T7 promoterl sequence
  source 1..18: mol_type = other DNA
taatacgactcactatag

(19) United States
(12) Patent Application Publication
(10) Pub. No.: US 2026/0199388 A1
(43) Pub. Date: Jul. 16, 2026

(54) HETERO-STRUCTURED RIBONUCLEIC ACID AND USE THEREOF

(71) Applicant: NA VACCINE INSTITUTE, Seoul (KR)

(72) Inventors: Dong Ho KIM, Seongnam-si (KR); Myung Soo KANG, Seoul (KR)

(73) Assignee: NA VACCINE INSTITUTE, Seoul (KR)

(21) Appl. No.: 19/436,030

(22) Filed: Dec. 30, 2025

Related U.S. Application Data
(63) Continuation of application No. 17/510,536, filed on Oct. 26, 2021, now Pat. No. 12,539,310.
Application No. 17/510,536 is a continuation-in-part of application No. PCT/KR2020/008623, filed on Jul. 1, 2020.

(30) Foreign Application Priority Data
Jul. 2, 2019 (KR) 10-2019-0079470
Nov. 26, 2019 (KR) 10-2019-0153760
Mar. 27, 2020 (KR) 10-2020-0037711

(51) Int. Cl. A61K 31/7105; A61K 39/00; A61P 35/00

(52) U.S. Cl. CPC A61K 31/7105; A61K 39/0011; A61P 35/00; A61K 2039/5156

(57) ABSTRACT

A hetero-structured RNA with two 3′-overhangs and use thereof as an immune stimulant are provided.

### CROSS-REFERENCE TO RELATED APPLICATIONS

This application is a continuation of application Ser. No. 17/510,536, filed Oct. 26, 2021, which is a continuation-in-part of International Patent Application No. PCT/KR2020/008623, filed on Jul. 1, 2020, which claims the benefit of Korean Patent Application Nos. 10-2019-0079470, filed on Jul. 2, 2019, 10-2019-0153760, filed on Nov. 26, 2019, and 10-2020-0037711, filed on Mar. 27, 2020, in the Korean Intellectual Property Office. The entire disclosure of each of these applications is hereby incorporated herein by reference for all purposes.

### SEQUENCE LISTING

In accordance with 37 CFR § 1.831-1.835 and 37 CFR § 1.77 (b)(5)(ii), the specification makes reference to a Sequence Listing submitted electronically as a .xml file named “560756US_REPLACEMENT_SEQUENCE_LISTING_ST26”. This .xml file was generated on Mar. 23, 2026 and is 197,065 bytes in size. The entire contents of the Sequence Listing are hereby incorporated herein by reference.

### BACKGROUND

### 1. Field

The present disclosure relates to a hetero-structured ribonucleic acid having two 3′-overhangs and use thereof.

### 2. Description of the Related Art

Toll-like receptor 3 (TLR3) is a protein that in humans is encoded by the TLR3 gene. TLR3 is a member of the toll-like receptor family of pattern recognition receptors of the innate immune system. TLR 3 is usually expressed on endosomes and detects double-stranded RNA (dsRNA) released from viruses during an invasion or necrotic cells during inflammation. Immune cells such as dendritic cells (DCs), B cells, and macrophages express TLR3. A variety of cancer cells also have been reported to express TLR3. Upon recognizing the dsRNA, TLR 3 stimulates the secretion of type I interferons and proinflammatory cytokines and activates DCs into mature antigen-presenting cells (APCs). The activation of DC by a TLR3 initiates an innate immune response and contributes to an adaptive immune response.

Polyinosinic: polycytidylic acid (referred to as “PolyIC”) is a dsRNA mimetics. PolyIC interacts with TLR3 on the endosomal membrane of B-cells, macrophages, and dendritic cells. PolyIC is structurally similar to dsRNA, present in some viruses, and is an “artificial” stimulant of TLR3. PolyIC may be considered a synthetic analog of dsRNA analog and is a standard tool for scientific research on the immune system. PolyIC is a mismatched double-stranded RNA, with one strand being a polymer of inosinic acid, the other a polymer of cytidylic acid.

PolyIC interaction with TLR3 leads to activate DCs. The significant disadvantages of PolyIC include high heterogeneity in length, thus less favorable pharmacokinetics, and increased toxicity. These weaknesses may have arisen during the manufacturing process.

Optimization of physicochemical properties of PolyIC has led to the generation of derivatives that have increased stability in body fluids such as PolyIC-L-lysine (PolyIC-L) or PolyIC-L-lysine-methylcellulose (PolyIC-LC), or reduced toxicity through reduced stability in body fluids such as Poly IC2U. Poly IC12U comprises Poly(I) and Poly(C) in which U is added to every 12 C. These PolyIC still has undefined chemical properties and very poor homogeneity, with subsequent unpredictable pharmacokinetics.

Thus, there is a need to develop a double-stranded RNA with more defined chemical properties and homogeneity with high immune-stimulating activity and the use thereof.

### SUMMARY

Provided is a hetero-structured RNA comprising heteropolymeric double-stranded RNA region having a first strand and a second strand and two single-stranded RNA regions, wherein the ssRNA regions are positioned at 3′-end of the first strand and 3′-end of the second strand.

Also provided is a method of producing the hsRNA comprising providing a template DNA molecule including a double-stranded DNA region and two RNA polymerase promoter sequences, wherein a first RNA polymerase promoter sequence is positioned at one end of the DNA region and a second RNA polymerase promoter sequence is positioned at the other end of the DNA region and the two promoters are oriented in opposing direction so that transcription from the first promoter proceeds toward the second promoter and transcription from the second promoter proceeds toward the first promoter; and incubating the template DNA molecule with RNA polymerase to produce the hsRNA, wherein the hsRNA comprises a heteropolymeric double-stranded RNA region having a first strand and a second strand and two single-stranded RNA regions, wherein the ssRNA regions are positioned at 3′-end of the first strand and 3′-end of the second strand.

Also provided is a pharmaceutical composition comprising the hsRNA.

Also provided is a method of treating a disease or disorder comprising administering to a subject in need of such treatment a therapeutically effective amount of the hsRNA.

Also provided is a method of enhancing an immune response to an antigen in a subject comprising administering to a subject in need of such treatment a therapeutically effective amount of the hsRNA.

### BRIEF DESCRIPTION OF THE DRAWINGS

These and/or other aspects will become apparent and more readily appreciated from the following description of the embodiments, taken in conjunction with the accompanying drawings in which:

FIG. 1 shows an in vivo screening result of an RNA-based adjuvant for activating DCs.

FIG. 2 shows the activity of DC in vivo according to the length of the hsRNA.

FIG. 3 shows the innate immunity activity marker and immune response result of the hsRNA.

FIG. 4 shows the physicochemical properties of NVT, one of the hsRNA.

FIG. 5 shows the pattern of innate immunity activity markers after subcutaneous injection of the hsRNA.

FIG. 6 shows that the NVT-adjuvanted OVA vaccine induces a Th1 polarized immune response more strongly than other adjuvants.

FIG. 7 shows the comparison of NVT and NVT II in an innate immune and adaptive immunity induction.

FIG. 8 shows the structure of the hsRNA.

FIG. 9 shows the immune response after intranasal administration of the whole inactivated influenza vaccine adjuvanted with (iPR8+NVT) or without (iPR8) the NVT, or NVT alone.

FIG. 10 relates to an effect of the adjuvant of the present invention on an efficacy and antigen dose saving of a commercial influenza vaccine (4IV (Vaxigrip®) by a hsRNA NVT.

FIG. 11 shows an induction in antibody IgG against a meningococcus vaccine (Menactra®, Sanofi) adjuvanted with NVT II.

FIG. 12 shows an effect of the NVT II adjuvant on immunogenicity restoration.

FIG. 13 shows an effect of the hsRNA alone on the arrest of melanoma growth.

FIG. 14 shows an effect of the hsRNA alone on the growth arrest of colorectal cancer and lung cancer.

FIG. 15 shows an inhibitory effect of the hsRNA alone on the growth arrest and metastasis of triple-negative breast cancer (TNBC).

FIG. 16 shows an inhibitory effect of NVT-adjuvanted OVA cancer vaccine administered intraperitoneally on the melanoma cell metastasis.

FIG. 17 shows an inhibitory effect of a cancer vaccine comprising the hsRNA and an OX40 antibody on primary and remote cancer growth.

FIG. 18 shows an inhibitory effect of the hsRNA in combination with a PD-1 antibody or OX40 antibody on melanoma growth.

### DETAILED DESCRIPTION

Hereinafter, embodiments and examples of the present application will be described in detail regarding the accompanying figures so that one of ordinary skill in the art to which the present invention pertains can easily practice. However, the present application may be implemented in various forms and is not limited to the embodiments and examples described herein.

Throughout the present specification, when a particular part “includes” a specific component, it means that the part can further comprise other components, rather than excluding other components unless otherwise stated.

The term “overhang” as used herein, refers to terminal non-base pairing nucleotide(s) resulting from one strand or region extending beyond the terminus of the complementary strand to which the first strand or region forms a duplex. The single-stranded region extending beyond the 3′-end of the duplex is referred to as a 3′-overhang.

According to an aspect of the present invention, a hetero-structured RNA (hsRNA) includes heteropolymeric double-stranded RNA (dsRNA) region having a first strand and a second strand and single-stranded RNA (ssRNA) regions, wherein the ssRNA regions are positioned at 3′-end of the first strand and 3′-end of the second strand. The hsRNA may be a set of the hsRNA comprising a plurality of the hsRNA molecules.

The hsRNA may act as a TLR3 ligand. The hsRNA may have 140 to 1682 nt, 200 to 1500 nt, 300 to 1000 nt, 400 to 900 nt, or 600 to 900 nt in length. The dsRNA region may act as a TLR 3 ligand. The dsRNA may have 106 to 1648 nt, 200 to 1500 nt, 300 to 1000 nt, 400 to 900 nt, or 600 to 900 nt in length.

The ssRNA region may have 1 to 100 nt, 1 to 90 nt, 1 to 80 nt, 1 to 70 nt, 1 to 60 nt, 1 to 50 nt, 1 to 40 nt, 1 to 30 nt, 1 to 20 nt, 1 to 20 nt, 1 to 15 nt, 1 to 10 nt, 1 to 8 nt, 1 to 6 nt, 2 to 100 nt, 2 to 90 nt, 2 to 80 nt, 2 to 70 nt, 2 to 60 nt, 2 to 50 nt, 2 to 40 nt, 2 to 30 nt, 2 to 20 nt, 2 to 10 nt, 2 to 8 nt, 2 to 6 nt, 3 to 100 nt, 3 to 90 nt, 3 to 80 nt, 3 to 70 nt, 3 to 60 nt, 3 to 50 nt, 3 to 40 nt, 3 to 30 nt, 3 to 20 nt, 3 to 10 nt, 3 to 8 nt, 3 to 6 nt, 15 to 80 nt, or 17 to 75 nt in length. For example, the ssRNA region may have 1 nt, 2 nt, 3 nt, 4 nt, 5 nt, 6 nt, 7 nt, 8 nt, 9 nt, or 10 nt in length.

Further, the hsRNA may not be a homo-polyribonucleotide. The hsRNA may not be a PolyIC, PolyIC-L-lysine, PolyIC-L-lysine-methylcellulose, poly (I:C12U), or combination thereof. The ssRNA regions may not or substantially not be complementary to each other.

In the hsRNA, the first strand may have a completely complementary nucleotide sequence with that of the second strand. The first strand may have completely complementary ribonucleotides without a gap with that of the second strand. The dsRNA region may not have a secondary structure such as stem-and-loop structure. The dsRNA region may not have a nick.

The hsRNA or dsRNA region is not designed to have RNAi or antisense inhibition activity. The hsRNA may comprise two strands of ssRNA, and each strand of the ssRNA may be a separate molecule.

The dsRNA region may have no more than 5%, 10%, 15%, 20%, 25%, 30%, 35%, or 40% sequence identity with a naturally-existing human gene. The hsRNA or dsRNA region may not encode a protein.

The dsRNA region may be derived from non-human organisms. The non-human organism may be a virus, prokaryotic cell, or eukaryotic cell. The prokaryotic cell may include bacterial cells. The dsRNA region may be derived from an artificial sequence, a vector sequence, a viral sequence, or a plant genome. The artificial sequence may consist of any partial sequences. The vector sequence may be the pDM-18T. The viral sequence may be a nucleotide sequence encoding Sacbrood virus VP1. The artificial sequence may be an arbitarily synthesized sequence. The plant genome may be a nucleotide sequence encoding tomato EPSP-1 protein. The eukaryotic cell may be a fungal cell such as yeast, plant cell, and animal cell. The hsRNA or dsRNA region may be an artificially synthesized or recombinant dsRNA. The hsRNA may be a dsRNA having two 3′-overhangs. The hsRNA may be an isolated dsRNA, which is not present in nature.

In the hsRNA, the ssRNA may be formed by contacting a pre-hsRNA with an endoribonuclease. The endoribonuclease may be an enzyme specifically cleaving the ssRNA region. The endoribonuclease may not be an enzyme cleaving dsRNA region. The endoribonuclease may be an enzyme specifically cleaving ssRNA region and does not or substantially not cleave dsRNA region. The ssRNA region may be formed by cleaving the ssRNA region with a longer length with endoribonuclease and has reduced length. The ssRNA region may be residual sequence after cleavage with the endoribonuclease. The endoribonuclease may be RNase 1. The RNase 1 is an endoribonuclease that specifically cleaves single-stranded RNA at G residues. It may cleave the phosphodiester bond between the 3′-guanylic residue and the 5′-OH residue of adjacent nucleotides. The reaction products may be 3′-GMP and oligonucleotides with a terminal 3′-GMP.

The ssRNA region may have UAUAG sequence at the 3′-end of each ssRNA region.

The hsRNA may be obtained by contacting a pre-hsRNA having two 3′-overhangs with endoribonuclease to reduce the length of the ssRNA region. The two ssRNA regions may have identical lengths.

The hsRNA may independently have triphosphate, diphosphate, or monophosphate at its 5′-end. For example, the hsRNA may have a triphosphate at its both 5′-ends. The hsRNA may independently have a hydroxyl group at its 3′-end. For example, the hsRNA may have hydroxyl groups at both 3′-ends. The first strand and the second strand are disposed on separate RNA molecules.

The hsRNA may further include a nucleotide sequence acting as a TLR7-like ligand or a TLR8-like ligand. The hsRNA may serve as a TLR7-like ligand or a TLR8-like ligand. The nucleotide sequence may be linked to the promoter sequence and positioned to form the nucleotide sequence-promoter-dsRNA region-promoter—the nucleotide sequence.

The hsRNA may be a double-stranded RNA comprising the following nucleotide sequence set, wherein one nucleotide sequence of the set base pairs with the other nucleotide sequence of the set to form a double-stranded RNA with two 3′-overhangs: SEQ ID NOs: 1 and 32, SEQ ID NOs: 2 and 33, SEQ ID NOs: 3 and 34, SEQ ID NOs: 4 and 35, SEQ ID NOs: 5 and 36, SEQ ID NOs: 6 and 37, SEQ ID NOs: 7 and 38, SEQ ID NOs: 8 and 39, SEQ ID NOs: 9 and 40, SEQ ID NOs: 10 and 41, SEQ ID NOs: 11 and 42, SEQ ID NOs: 12 and 43, SEQ ID NOs: 13 and 44, SEQ ID NOs: 14 and 45, SEQ ID NOs: 15 and 46, SEQ ID NOs: 16 and 47, SEQ ID NOs: 17 and 48, SEQ ID NOs: 18 and 49, SEQ ID NOs: 19 and 50, SEQ ID NOs: 20 and 51, SEQ ID NOs: 21 and 52, SEQ ID NOs: 22 and 53, SEQ ID NOs: 23 and 54, SEQ ID NOs: 24 and 55, SEQ ID NOs: 25 and 56, SEQ ID NOs: 26 and 57, SEQ ID NOs: 27 and 58, SEQ ID NOs: 28 and 59, SEQ ID NOs: 29 and 60, SEQ ID NOs: 30 and 61, or SEQ ID NOs: 31 and 62.

The hsRNA may be a double-stranded RNA obtained by contacting a dsRNA with two 3′-overhangs with endoribonuclease to cleave the dsRNA with two 3′-overhangs and thus reduce the length of the 3′-overhang. The endoribonuclease may be RNase T1.

The hsRNA may be a double-stranded RNA comprising the following nucleotide sequence set, wherein one nucleotide sequence of the set base pairs with the other nucleotide sequence of the set to form a double-stranded RNA with two 3′-overhangs: the nucleotide sequence of SEQ ID NO: 1 from nucleotide position 1 to 111 and the nucleotide sequence of SEQ ID NO: 32 from nucleotide position 1 to 111, the nucleotide sequence of SEQ ID NO: 2 from nucleotide position 1 to 161 and the nucleotide sequence of SEQ ID NO: 33 from nucleotide position 1 to 161, the nucleotide sequence of SEQ ID NO: 3 from nucleotide position 1 to 211 and the nucleotide sequence of SEQ ID NO: 34 from nucleotide position 1 to 211, the nucleotide sequence of SEQ ID NO: 4 from nucleotide position 1 to 311 and the nucleotide sequence of SEQ ID NO: 35 from nucleotide position 1 to 311, the nucleotide sequence of SEQ ID NO: 5 from nucleotide position 1 to 411 and the nucleotide sequence of SEQ ID NO: 36 from nucleotide position 1 to 411, the nucleotide sequence of SEQ ID NO: 6 from nucleotide position 1 to 511 and the nucleotide sequence of SEQ ID NO: 37 from nucleotide position 1 to 511, the nucleotide sequence of SEQ ID NO: 7 from nucleotide position 1 to 611 and the nucleotide sequence of SEQ ID NO: 38 from nucleotide position 1 to 611, the nucleotide sequence of SEQ ID NO: 8 from nucleotide position 1 to 711 and the nucleotide sequence of SEQ ID NO: 39 from nucleotide position 1 to 711, the nucleotide sequence of SEQ ID NO: 9 from nucleotide position 1 to 811 and the nucleotide sequence of SEQ ID NO: 40 from nucleotide position 1 to 811, the nucleotide sequence of SEQ ID NO: 10 from nucleotide position 1 to 111 and the nucleotide sequence of SEQ ID NO: 41 from nucleotide position 1 to 111, the nucleotide sequence of SEQ ID NO: 11 from nucleotide position 1 to 161 and the nucleotide sequence of SEQ ID NO: 42 from nucleotide position 1 to 161, the nucleotide sequence of SEQ ID NO: 12 from nucleotide position 1 to 211 and the nucleotide sequence of SEQ ID NO: 43 from nucleotide position 1 to 211, the nucleotide sequence of SEQ ID NO: 13 from nucleotide position 1 to 311 and the nucleotide sequence of SEQ ID NO: 44 from nucleotide position 1 to 311, the nucleotide sequence of SEQ ID NO: 14 from nucleotide position 1 to 411 and the nucleotide sequence of SEQ ID NO: 45 from nucleotide position 1 to 411, the nucleotide sequence of SEQ ID NO: 15 from nucleotide position 1 to 511 and the nucleotide sequence of SEQ ID NO: 46 from nucleotide position 1 to 511, the nucleotide sequence of SEQ ID NO: 16 from nucleotide position 1 to 611 and the nucleotide sequence of SEQ ID NO: 47 from nucleotide position 1 to 611, the nucleotide sequence of SEQ ID NO: 17 from nucleotide position 1 to 711 and the nucleotide sequence of SEQ ID NO: 48 from nucleotide position 1 to 711, the nucleotide sequence of SEQ ID NO: 18 from nucleotide position 1 to 811 and the nucleotide sequence of SEQ ID NO: 49 from nucleotide position 1 to 811, the nucleotide sequence of SEQ ID NO: 19 from nucleotide position 1 to 324 and the nucleotide sequence of SEQ ID NO: 50 from nucleotide position 1 to 324, the nucleotide sequence of SEQ ID NO: 20 from nucleotide position 1 to 402 and the nucleotide sequence of SEQ ID NO: 51 from nucleotide position 1 to 402, the nucleotide sequence of SEQ ID NO: 21 from nucleotide position 1 to 471 and the nucleotide sequence of SEQ ID NO: 52 from nucleotide position 1 to 471, the nucleotide sequence of SEQ ID NO: 22 from nucleotide position 1 to 593 and the nucleotide sequence of SEQ ID NO: 53 from nucleotide position 1 to 593, the nucleotide sequence of SEQ ID NO: 23 from nucleotide position 1 to 669 and the nucleotide sequence of SEQ ID NO: 54 from nucleotide position 1 to 669, the nucleotide sequence of SEQ ID NO: 24 from nucleotide position 24 to 738 and the nucleotide sequence of SEQ ID NO: 55 from nucleotide position 1 to 738, the nucleotide sequence of SEQ ID NO: 25 from nucleotide position 1 to 827 and the nucleotide sequence of SEQ ID NO: 56 from nucleotide position 1 to 827, the nucleotide sequence of SEQ ID NO: 26 from nucleotide position 1 to 890 and the nucleotide sequence of SEQ ID NO: 57 from nucleotide position 1 to 890, the nucleotide sequence of SEQ ID NO: 27 from nucleotide position 1 to 1037 and the nucleotide sequence of SEQ ID NO: 58 from nucleotide position 1 to 1037, the nucleotide sequence of SEQ ID NO: 28 from nucleotide position 1 to 1158 and the nucleotide sequence of SEQ ID NO: 59 from nucleotide position 1 to 1158, the nucleotide sequence of SEQ ID NO: 29 from nucleotide position 1 to 1653 and the nucleotide sequence of SEQ ID NO: 60 from nucleotide position 1 to 1653, the nucleotide sequence of SEQ ID NO: 30 from nucleotide position 1 to 429 and the nucleotide sequence of SEQ ID NO: 61 from nucleotide position 1 to 429, or the nucleotide sequence of SEQ ID NO: 31 from nucleotide position 1 to 429 and the nucleotide sequence of SEQ ID NO: 62 from nucleotide position 1 to 429.

The hsRNAs may be homogenous in length and structure. The hsRNA may be stable at room temperature.

According to another aspect of the present invention, a method of producing a hsRNA comprising providing a template DNA molecule including a double-stranded DNA region and two RNA polymerase promoter sequences, wherein a first RNA polymerase promoter sequence is positioned at one end of the DNA region and a second RNA polymerase promoter sequence is positioned at the other end of the DNA region and the two promoters are oriented in opposing direction so that transcription from the first promoter proceeds toward the second promoter and transcription from the second promoter proceeds toward the first promoter; and incubating the template DNA molecule with RNA polymerase to produce the hsRNA,
wherein the hsRNA comprises a heteropolymeric double-stranded RNA region having a first strand and a second strand and single-stranded RNA regions, wherein the ssRNA regions are positioned at 3′-end of the first strand and 3′-end of the second strand.

Regarding the hsRNA, it is the same as described above.

In the step of providing, the double-stranded DNA region may have 106 to 1648 nt, 200 to 1500 nt, 300 to 1000 nt, 400 to 900 nt, or 600 to 900 nt in length. The template DNA molecule may have 140 to 1682 nt, 200 to 1500 nt, more 300 to 1000 nt, 400 to 900 nt, or 600 to 900 nt in length. The template DNA molecule may further comprise an additional sequence linked to the promoter. The additional sequence may be a ligand sequence such as TLR7 ligand-like sequence or TLR8 ligand-like sequence.

The template DNA molecule may be produced by a known method. The template DNA molecule may be produced by a nucleic acid amplification method such as PCR, by using a recombinant cell or artificial synthesis. The template DNA molecule may be produced by culturing a host cell comprising the template DNA molecules. The method may include introducing the template DNA molecule into a host cell, culturing the host cell in a medium, and isolating the template DNA molecule from the culture. The host cell may be a bacterial cell such as E. coli. The template DNA molecule may be introduced into the host cell in itself or via a vector such as a plasmid or viral vector. The template DNA molecule may be a double-stranded DNA with blunt ends and having double-stranded promoter sequences at both ends.

The first RNA polymerase promoter sequence may have an identical sequence with that of the second RNA polymerase promoter sequence. The RNA polymerase promoter may include a restriction site for endoribonuclease. The RNA polymerase may be an RNA polymerase of a bacteriophage. The RNA polymerase promoter may be T7 promoter, T3 promoter, SP6 promoter, K11 promoter, or B14 promoter. The promoter sequence may be a double-stranded RNA sequence. The promoter sequence may be a promoter recognized by single species of RNA polymerase.

In the step of transcription, single species of RNA polymerase may be used in the reaction. The reaction may be conducted in suitable conditions to allow the transcription reaction to proceed. The transcription may be in vitro transcription (IVT) or in vivo transcription. The in vitro transcription may be conducted by transforming an appropriate IVT template DNA into an E. coli cell expressing appropriate polymerases.

For in vitro transcription, the incubating may be performed in vitro. The incubation may result in vitro transcription of the template DNA molecule. The transcription may proceed bidirectionally from each of the two promoters, yielding two RNA strands, i.e., upper and lower strands, which are almost complementary to each other except a short stretch of non-complementary nucleotides at 3′ ends. The two RNA strands may spontaneously simultaneously associate by base-pairing to form a double-stranded RNA with two 3′-overhangs during the in vitro transcription.

The template DNA molecule may not include a transcription termination sequence. The template DNA for PCR may be cut to be able to get linear template DNA. The transcription termination may be a run-off termination.

The method may further include incubation of the IVT product to DNase so as to cleave the residual template DNAs.

The method may further comprise incubating the IVT product with endoribonuclease to cleave a single-stranded RNA region. The endoribonuclease may be endoribonuclease specifically cleaves single-stranded RNA, but not substantially a double-stranded RNA. The endoribonuclease may be RNase T1.

The method may further comprise isolating the hsRNA from the reaction product. The isolated hsRNAs may be homogenous in length and may have defined physicochemical properties. The isolated hsRNAs may have an identical length of 3′-overhangs. The length of 3′-overhangs may be 1 to 10 nt, 2 to 8 nt, 3 to 7 nt, 2 to 6 nt, or 5 nt. The 3′-overhangs may have UAUAG sequences at their 3′-ends. The isolated hsRNAs may not have nicks. The isolated hsRNAs may have ligand sequences linked to the 3′-end of the promoter sequence. The ligand sequence may be TLR7 or TLR8-like ligand. TLR7 or TLR8-like ligand may include G or U nucleotide such as GUU repeat.

In an embodiment, template DNA molecules capable of directing transcription to a target RNA may be flanked by a T7 promoter at both 5′ ends and be inserted into a vector. If necessary, an additional nucleic acid sequence such as a TLR7 or TLR8 ligand sequence may be added to the 5′ end of the T7 promoter sequence. The template DNA molecules may be amplified by a method such as PCR to obtain template DNA molecules for transcription. Bidirectional in vitro transcription may generate two strands of heteropolymeric single-stranded RNAs with perfect complementarity regions at almost the same molar ratio during the transcription. The two stands may spontaneously form hsRNA at room temperature. Thus, hsRNAs with high homogeneity and defined structure may be obtained. The hsRNA may or may not encode a protein.

According to another aspect of the invention, a pharmaceutical composition comprising the hsRNA is provided.

The pharmaceutical composition may include a pharmaceutically acceptable carrier, excipient, or stabilizer.

The term “pharmaceutically acceptable” used herein refers that the respective material does not show any biological or otherwise undesirable effects when administered to an individual and does not interact in a deleterious manner with any of the other components of the pharmaceutical composition (such as e.g. the pharmaceutically active ingredient) in which it is contained.

The pharmaceutical composition may be formulated for parenteral administration, such as intravenous, intramuscular, subcutaneous injection or intravenous infusion may, for example, be sterile solutions, suspensions, dispersions, emulsions, or powders which comprise the active ingredient and which are suitable, optionally after a further dissolution or dilution step, for infusion or injection. Suitable carriers or diluents may include sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol, as well as mineral oils, animal oils and vegetable oils, for example, peanut oil, soybean oil, as well as suitable mixtures thereof.

The hsRNA is described as above.

The pharmaceutical composition may be for preventing or treating an infection, cancer, or cancer-related diseases.

The infection may be a viral or bacterial infection. The viral infection may be an infection with an influenza virus, human parainfluenza virus, coronavirus, betacoronavirus, respiratory syncytial virus, measles virus, human metapneumovirus, enteroviruses, rhinoviruses, polioviruses, coxsackieviruses, or echoviruses. The betacoronavirus may include MERS-COV, SARS-COV, SARS-COV-2, HCoV-OC43, HCOV-229E, HCOV-NL63, HCOV-NL, HCOV—NH, HCoV-HKU1, or a combination thereof. SARS-COV-2 is also called COVID-19. The influenza virus may include influenza viruses A, B, C, and D. The bacteria may include gram-negative and gram-positive bacteria. The bacteria may be of the following genus: Streptococcus, Escherichia, Staphylococcus, Klebsiella, Pseudomonas, or Neisseria. The bacteria may be E. coli, S. aureus, P. aeruginosa, S. pneumonia, K. pneumonia, or N. meningitides. The N. meningitidis may be of serotype A, B, C, W, Y, X, or a combination thereof. The serotype B also refers to Meningococcus Serogroup B (MenB). The serotype W may be serotype W-135.

Cancer may include a solid tumor or blood cancer. Cancer may include squamous cell carcinoma, myeloma, skin cancer, lung cancer, small cell lung cancer, non-small cell lung cancer, colorectal cancer, glioma, Hodgkin's lymphoma, non-Hodgkin's lymphoma, acute myeloid leukemia (AML), multiple myeloma, gastrointestinal cancer, kidney cancer, ovarian cancer, liver cancer, lymphoblastic leukemia, lymphocytic leukemia, colorectum cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatocellular cancer, breast cancer, colon carcinoma, head and neck cancer, pancreas cancer, a combination thereof, or metastatic cancer thereof.

The pharmaceutical composition may be for providing an immune response to an antigen, alone or in combination with an antigen. Thus, the pharmaceutical composition may be an adjuvant composition. The pharmaceutical composition may be for providing an enhanced immune response to an antigen.

The pharmaceutical composition may further include an antigen or therapeutic agent, or adjuvant.

The antigen may be a known antigen. The antigen may be a protein of a pathogen, a recombinant protein, a subunit, a glycoprotein, a peptide, a polysaccharide, a lipopolysaccharide, a polynucleotide, an inactivated or live-attenuated organism, or a combination thereof. The antigen may be a viral antigen, bacterial antigen, or tumor-associated antigen, including an inactivated cancer cell. The antigen may include a live attenuated microorganism that causes known disease, an antigen that is an inactivated or killed microorganism that causes known diseases, an antigen that is an inactivated toxin that is produced by a microorganism that causes known diseases, or an antigen that is a subunit or a conjugate of a subunit of a microorganism that causes known diseases.

The therapeutic agent may be suitably selected in consideration of the disease and the conditions of the subject to be treated. The therapeutic agent may be an anti-cancer drug or immune stimulant. The therapeutic agent may be an immune checkpoint inhibitor. The immune checkpoint inhibitor may be a PD-1 antibody, PDL1 antibody, CTLA4 antibody, OX40 antibody, or a combination thereof.

The immune checkpoint inhibitor may be an agonist antibody. The OX40 antibody may be an OX40 agonist antibody, and the PD-1 antibody may be a PD-1 antagonist antibody. The OX40 antibody may be a monoclonal antibody or an antigen-binding fragment thereof, specifically binding to OX40. The PD-1 antibody may be a monoclonal antibody or an antigen-binding fragment thereof, specifically binding to PD-1. The OX40 antibody or PD-1 antibody may be human, a humanized antibody, a chimeric antibody, or a mouse antibody and may comprise a human constant region. The human constant region may be IgG1, IgG2, IgG3, or IgG4 constant region. The human constant region may be IgG1 or IgG4 constant region. In one embodiment, the antigen-binding fragment may be Fab, Fab′-SH, F(ab′) 2, scFv, or Fv fragment. Examples of anti-human PD-1 monoclonal antibodies that may be used as the PD-1 antagonist antibody include pembrolizumab, nivolumab, avelumab, and pidilizumab.

The adjuvant may be an oil-in-water emulsion adjuvant, aluminum salt, Freund adjuvant, 1,2-dioleoyl-3-trimethylammonium-propane (DOTAP), or a combination thereof. The oil-in-water emulsion adjuvant may be a squalene oil-in-water emulsion.

The pharmaceutical composition may be formulated as an oral or injectable solution. The pharmaceutical composition may be formulated in an oil-in-water emulsion. The pharmaceutical composition may be administered by intravenous injection, intratumoral injection, subcutaneous injection, intraperitoneal injection, intracranial injection, intrathecal injection, intrastriatal injection, intranasal injection, or intracerebroventricular injection.

The pharmaceutical composition may include the hsRNA in the amount of about 5 ug to 150 mg/single dose. The pharmaceutical composition may be aqueous solutions or suspensions. The pharmaceutical composition may be buffered nucleic acid solutions, such as solutions including the hsRNA in a suitable concentration for example, from 0.001 to 400 mg/ml, from 0.005 to 200 mg/ml, 0.01 to 200 mg/ml, 1.0-100 mg/ml, 1.0 mg/ml or 100 mg/ml and an aqueous buffer such as: phosphate-buffered saline.

The strongly negatively charged RNA such as the hsRNA might effectively receive many large antigens to make a nanocomplex comparable to a virus-like particle (VLP). The nanocomplex encapsulating the antigen in an appropriate formulation may be captured by dendritic cells or macrophages, resulting in an effective presentation of antigens to B cells and T cells.

According to another aspect of the invention, a method of treating a disease or disorder comprising administering to a subject in need of such treatment a therapeutically effective amount of the hsRNA is provided.

According to another aspect of the invention, a method of enhancing an immune response to an antigen in a subject comprising administering to a subject in need of such treatment a therapeutically effective amount of the hsRNA is provided.

The shRNA is described as above. The administering may include administering via oral or parenteral route. The parenteral route may include intravenous, intracranial, intrathecal, intrastriatal, intracerebroventricular, intranasal, intra-tumoral, intramuscular, intraperitoneal, or mucosal route.

The term “therapeutically effective amount” used herein may refer to an amount sufficient to enhance the immune response in the subject. The “therapeutically effective amount” may be 150 ug to 150 mg/kg body weight/day.

The disease or disorder may include infection, cancer, or cancer-related diseases. The infection may be a viral or bacterial infection. The viral infection may influenza virus, human parainfluenza virus, coronavirus, betacoronavirus, respiratory syncytial virus, measles virus, human metapneumovirus, enteroviruses, rhinoviruses, polioviruses, coxsackieviruses, or echoviruses. The betacoronavirus may include MERS-COV, SARS-COV, SARS-COV-2, HCoV-OC43, HCOV-229E, HCoV-NL63, HCoV-NL, HCOV—NH, HCOV-HKU1, or a combination thereof. SARS-COV-2 is also called COVID-19. The influenza virus may include influenza viruses A, B, C, and D.

The bacteria may include gram-negative and gram-positive bacteria. The bacteria may be of the following genus: Streptococcus, Escherichia, Staphylococcus, Klebsiella, Pseudomonas, or Neisseria. The bacteria may be E. coli, S. aureus, P. aeruginosa, S. pneumonia, K. pneumonia, or N. meningitides. The N. meningitidis may be of serotype A, B, C, W, Y, X, or a combination thereof. The serotype B also refers to Meningococcus Serogroup B (MenB). The serotype W may be serotype W-135.

Cancer may include squamous cell carcinoma, myeloma, skin cancer, lung cancer, small cell lung cancer, non-small cell lung cancer, colorectal cancer, glioma, Hodgkin's lymphoma, non-Hodgkin's lymphoma, acute myeloid leukemia (AML), multiple myeloma, gastrointestinal cancer, kidney cancer, ovarian cancer, liver cancer, lymphoblastic leukemia, lymphocytic leukemia, colorectum cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatocellular cancer, breast cancer, colon carcinoma, head and neck cancer, pancreas cancer or metastatic cancer thereof.

The immune response may include increased expression of a stimulatory cofactor such as CD40, and CD86 and MHCII in cells such as CD11c+DC cells, inducing secretion of IL-6, IL-12, TNF-α, inducing T-bet, and interferon-γ, increasing Th1 cell and Th2 cell response including inducing the production of IgG2a specific to an antigen and IgG1 specific to an antigen.

The method may further comprise administering an antigen or therapeutic agent to the subject.

The antigen may be a known antigen. The antigen may be a protein of a pathogen, a recombinant protein, a subunit, a glycoprotein, a peptide, a polysaccharide, a lipopolysaccharide, a polynucleotide, an inactivated or live-attenuated organism, or a combination thereof. The antigen may be a viral antigen, bacterial antigen, or tumor-associated antigen, including an inactivated cancer cell. The antigen may include a live attenuated microorganism that causes known disease, an antigen that is an inactivated or killed microorganism that causes known diseases, an antigen that is an inactivated toxin that is produced by a microorganism that causes known diseases, or an antigen that is a subunit or a conjugate of a subunit of a microorganism that causes known diseases. The antigen may be a trivalent seasonal influenza vaccine, a tetravalent seasonal influenza vaccine, an inactivated influenza vaccine, or a meningococcus vaccine. The meningococcus vaccine may include Neisseria meningitidis groups A, C, Y, and W-135. The hsRNA may exhibit a protective effect against viruses of allogeneic or heterologous subtypes.

Therapeutic agents may be suitably selected in consideration of the disease and the conditions of the subject to be treated. The therapeutic agent may be an anti-cancer drug or immune stimulant. The therapeutic agent may be a checkpoint inhibitor. The checkpoint inhibitor may be a PD-1 antibody, PDL1 antibody, CTLA4 antibody, OX40 antibody, or a combination thereof.

The subject may be a mammalian animal, including a human.

The hsRNA of the present invention may provide an improved immune response in a subject with high homogeneity and defined structure.

In addition, the antigen in complex with the hsRNA of the present invention may be delivered into the APCs to present the antigen to T cells and B cells efficiently. Furthermore, the hsRNA of the present invention may reduce antigen dose and induce protective immunity against viral or bacterial infection and cancer.

As a result, the hsRNA may enhance an adaptive immunity against an antigen, reduced the required amount of antigen in the vaccine, improved the antigen-specific Th1-polarized cross-protective response, enhance innate immunity, or a combination thereof. The hsRNA may be used alone or in combination with other antigens or therapeutic agents.

The hsRNA, alone or in combination with other anti-cancer drugs such as immune checkpoint inhibitors, may prevent or treat primary or secondary cancer. The immune checkpoint inhibitor may include antibodies to PD-1, PDL1, or OX40. The hsRNA may convert refractory cancer to an immune checkpoint inhibitor into responsive cancer.

Table 1 shows examples of hsRNAs. The hsRNA may include two single-stranded RNAs having the nucleotide sequence shown in Table 1 below, respectively. The two strands, i.e., two single-stranded RNA sequences, form a double-stranded RNA having two 3′-overhangs by base pairing between the first strand and the second strand. The nucleotide sequences of the double-stranded region of the hsRNA are also shown in

TABLE 1

| | Sequence of hsRNA | | |
|---|---|---|---|
| | The sequence of | The sequence of | The sequence of |
| | the first strand: | the second strand: | dsRNA region: |
| Name | SEQ ID NO | SEQ ID NO | SEQ ID NO |
| NA1001 | 1 | 32 | 63 |
| NA1501 | 2 | 33 | 64 |
| NA2001 | 3 | 34 | 65 |
| NA3001 | 4 | 35 | 66 |
| NA4001 | 5 | 36 | 67 |
| NA5001 | 6 | 37 | 68 |
| NA6001 | 7 | 38 | 69 |
| NA7001 | 8 | 39 | 70 |
| NA8001 | 9 | 40 | 71 |
| NA1002 | 10 | 41 | 72 |
| NA1502 | 11 | 42 | 73 |
| NA2002 | 12 | 43 | 74 |
| NA3002 | 13 | 44 | 75 |
| NA4002 | 14 | 45 | 76 |
| NA5002 | 15 | 46 | 77 |
| NA6002 | 16 | 47 | 78 |
| NA7002 | 17 | 48 | 79 |
| NA8002 | 18 | 49 | 80 |
| R1 | 19 | 50 | 81 |
| R2 | 20 | 51 | 82 |
| R3 | 21 | 52 | 83 |
| R4 | 22 | 53 | 84 |
| R5 | 23 | 54 | 85 |
| R6 | 24 | 55 | 86 |
| R7 | 25 | 56 | 87 |
| R8 | 26 | 57 | 88 |
| R9 | 27 | 58 | 89 |
| R10 | 28 | 59 | 90 |
| R11 | 29 | 60 | 91 |
| NVT | 30 | 61 | 92 |
| NVT4 | 31 | 62 | 93 |

The hsRNAs of NA1001, NA1501, NA2001, NA3001, NA4001, NA5001, NA6001, NA7001, and NA8001 are derived from the sequences of pDM-18T vector (group 1), for example SEQ ID NO: 96. The hsRNAs of NA1002, NA1502, NA2002, NA3002, NA4002, NA5002, NA6002, NA7002, and NA8002 are derived from the sequences encoding tomato 5-enolpyruvylshikimate 3-phosphate synthase-1 (EPSP-1) protein (group 2), for example SEQ ID NO: 97. The hsRNAs of R1, R2, R3, R4, R5, R6, R7, R8, R9, R10, and R11 are derived from the sequences of an artificial sequence (group 3), for example SEQ ID NO: 98. The hsRNAs of NVT and NVT4 are derived from Chinese Sacbrood virus strain BJ 2012, VP1 gene (group 4), for example SEQ ID NO: 95. Table 2 shows the length of hsRNA, dsRNA region, first 3′-overhang and second 3′-overhang. The length of hsRNA includes the length of the dsRNA region, the length of the first 3′-overhang, and the length of the second 3′-overhang.

TABLE 2

| | Length (nt) | | | |
|---|---|---|---|---|
| Name | hsRNA | dsRNA | First 3′-overhang | Second 3′-overhang |
| NA1001 | 140 | 106 | 17 | 17 |
| NA1501 | 190 | 156 | 17 | 17 |
| NA2001 | 240 | 206 | 17 | 17 |
| NA3001 | 340 | 306 | 17 | 17 |
| NA4001 | 440 | 406 | 17 | 17 |
| NA5001 | 540 | 506 | 17 | 17 |
| NA6001 | 640 | 606 | 17 | 17 |
| NA7001 | 740 | 706 | 17 | 17 |
| NA8001 | 840 | 806 | 17 | 17 |
| NA1002 | 140 | 106 | 17 | 17 |
| NA1502 | 190 | 156 | 17 | 17 |
| NA2002 | 240 | 206 | 17 | 17 |
| NA3002 | 340 | 306 | 17 | 17 |
| NA4002 | 440 | 406 | 17 | 17 |
| NA5002 | 540 | 506 | 17 | 17 |
| NA6002 | 640 | 606 | 17 | 17 |
| NA7002 | 740 | 706 | 17 | 17 |
| NA8002 | 840 | 806 | 17 | 17 |
| R1 | 353 | 319 | 17 | 17 |
| R2 | 431 | 397 | 17 | 17 |
| R3 | 500 | 466 | 17 | 17 |
| R4 | 622 | 588 | 17 | 17 |
| R5 | 698 | 664 | 17 | 17 |
| R6 | 767 | 733 | 17 | 17 |
| R7 | 856 | 822 | 17 | 17 |
| R8 | 919 | 885 | 17 | 17 |
| R9 | 1066 | 1032 | 17 | 17 |
| R10 | 1187 | 1153 | 17 | 17 |
| R11 | 1682 | 1648 | 17 | 17 |
| NVT | 533 | 424 | 51 | 58 |
| NVT4 | 568 | 424 | 75 | 69 |

The hsRNA may activate immune cells, including dendritic cells, neutrophils, B cells, macrophages, T cells, mast cells, natural killer cells, or a combination thereof.

The hsRNA may enhance an immune response to an antigen. Thus, when the hsRNA is used in combination with an antigen as an adjuvant, the amount of the antigen to induce sufficient immune response may be reduced compared to that an antigen is used alone, i.e., without the hsRNA. A sufficient immune response may be an immune response to prevent or treat an infection or cancer. The amount of the antigen may be reduced ½ or less, ⅓ times or less, ⅕ or less, 1/10 or less, 1/25 or less, 1/50 or less, or 1/100 or less.

The hsRNA may enhance, maintain, or restore the antigenicity of an antigen when the antigen is used with the hsRNA.

Hereinafter, the present invention will be described in more detail through the working examples. However, the following examples are provided only for the purpose of illustration, and the scope of the present invention is not limited thereto.

### Example 1. hsRNA Preparation and DC Activation by the hsRNA

1. Preparation of hsRNAs Having Two 3′-Overhangs

The hsRNAs shown in Tables 1 and 2 are prepared as follows.

A template DNA molecule comprising a template DNA region and T7 promoter sequences linked to both 5′-ends of the template DNA region was prepared. The template DNA region has the nucleotide sequence of SEQ ID NO: 96, 97, 95, and 98, respectively, which corresponds to the nucleotide sequence of pDM-18T vector, tomato EPSP-1 gene, Sacbrood virus VP1 gene, and an artificial sequence and T7 promoter has the nucleotide sequence of SEQ ID NO: 97. The template DNA molecule was ligated to PUC19 vector (Thermo Fisher, Cat SD0061) digested with Smal, to form a recombinant vector comprising the template DNA molecule. The recombinant vector was introduced into E. coli DH5a by a transformation. The transformed cells were grown in LB/Amp medium. The cells were isolated from the supernatant of the culture. The cells were lysed by using an alkaline solution, and the recombinant vector was isolated by using Qiagen® midi prep kit.

The template DNA molecule was amplified by a PCR using the recombinant vector as a template. The obtained template DNA molecule was used in in vitro transcription (IVT) using T7 polymerase as a template. In the PCR, an oligonucleotide set, wherein each oligonucleotide has a complementary sequence to a target to be amplified and the T7 promoter sequence at its 5′-end was used as primer set. PCR was conducted at 95° C., and 5 minutes, and 35 thermal cycles of 95° C., and 30 seconds and 60° C., 30 seconds and 72° C., 1 minute. The reaction mixture was incubated at 72° C. and 5 minutes.

In vitro transcription was conducted using the MEGAscript™ T7 Transcription Kit (Thermo Fisher, cat AMB13345) according to the manufacturer's′ protocols. The amplified template DNA molecule was used as a template. 1 ml reaction mixture comprising 10 ug linearized Template DNA, 75 mM NTP, 90 mM Tris base, 90 mM Boric acid, 2 mM EDTA, and T7 RNA polymerase 50 ul was incubated at 37° C. for 4 hours. The obtained reaction mixture was incubated at 80° C. for 20 minutes and cooled to room temperature for 30 minutes. The first single-strand RNA and the second single-strand RNA are simultaneously synthesized from each of the template DNA strands and hybridizes to form double-stranded RNAs having two 3′-overhangs.

The reaction mixture was centrifuged at 20° C., 4000 rpm for 3 minutes to remove white precipitation and obtain a supernatant solution. DNase I was added to the supernatant solution, and the resultant solution was incubated at 37° C. for 13 hours. The hsRNA was isolated from the reaction solution. As a result, 31 hsRNAs shown in Tables 1 and 2 were obtained. These hsRNAs have 3′-overhangs at both ends, 29 of these 31 hsRNA having 17 nt and in length at each 3′-end.

Further, RNase T1 was added to the DNase I-treated reaction mixture, and the resultant mixture was incubated at 37° C. for 2 hours. Then, the reaction mixture was incubated at 80° C. for 10 minutes and cooled to room temperature for 30 minutes. Then, the hsRNA having 3′-overhangs with reduced length was isolated by a nucleic acid precipitation method using isopropanol.

The isolated hsRNA has 3′-overhangs with 5 nt in length, which is shorter than 12 nt compared to the DNase I-treated hsRNA with 17 nt in length. These hsRNAs have UAUAG sequence at its 3′-end of the 3′-overhang. The hsRNA may have phosphate at its 5′-end and a hydroxyl group at its 3′-end. The phosphate may be triphosphate, diphosphate, or monophosphate. The hsRNA may not have 5′-cap.

2. Dendritic Cells Activation by the hsRNA

The present example demonstrates that the hsRNA activates an innate immunity in C57BL/6 mice when the hsRNA is injected via peritoneal injection. In this example, the hsRNA was solubilized in PBS (pH 7.2), and the hsRNA in PBS was injected into the mouse via peritoneal injection. PolyIC (InvivoGen, Poly(I:C) (HMW) VacciGrade™, cat #, vac-pic) was used as a positive control.

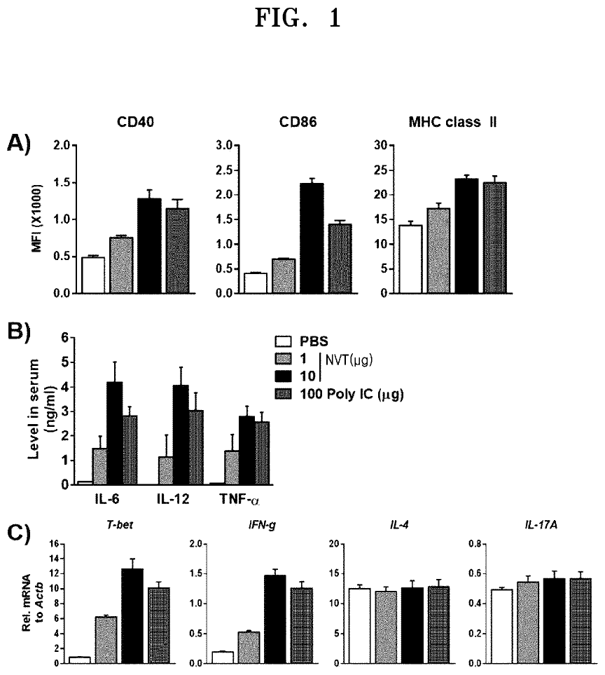

FIG. 1 shows an in vivo screening result of an hsRNA for activating DCs. Each data refer to an average value of three independent experiments.

As shown in A of FIG. 1, the NVT comprising the sequences of SEQ ID NOs: 30 and 61 followed by RNase T1 treatment increases expression of stimulatory cofactors CD40, and CD86, and MHC II on cell surface of CD11c+DC cells from spleen as measured by mean fluorescence intensity (MFI). MFI levels for CD40, CD86, and MHC II of the NVT 10 μg injection were equal or higher than that of the positive control, PolyIC 100 μg injection. This indicates that the NVT induces differentiation of DC maturation. The MFI was measured by flow cytometry analysis for DCs isolated from spleen.

As shown in B of FIG. 1, the NVT increases the serum level of IL-6, IL-12 and TNF-α compared to that of the positive control, PolyIC. This indicates that the NVT increases the secretion of IL-6, IL-12, and TNF-α in serum compared to that of the positive control. The serum level of IL-6, IL-12, and TNF-α was measured by a Sandwich ELISA methods.

As shown in C of FIG. 1, the NVT increases mRNA level of T-bet and IFN-γ, but does not increase expression of IL-4 and IL-17A compared to that of the positive control, PolyIC. T-bet and IFN-γ are known as Th1 cell markers and cytokine, respectively. IL-4 and IL-17A are known as Th2 and Th7 cell cytokine, respectively. Total RNA from indicated treatment was isolated using RNAzol® RT (Sigma Aldrich). The level of each mRNA was determined by qRT-PCR and relative abundance to beta actin mRNA was obtained.

These data indicate the hsRNA strongly activates naïve CD4 T cells to Th1 CD4 T cells as compared to that of the positive control.

### Example 2. DC Activation Depends on the Length of the hsRNA

The hsRNAs having varying length dsRNA region and constant length two 3′-overhangs were injected into C57BL/6 mice via peritoneal injection as shown Example 1. 17 hsRNAs shown in Tables 1 and 2, i.e., NA1001, NA1501, NA2001, NA3001, NA4001, NA5001, NA6001, NA8001, NA1002, NA1502, NA2002, NA3002, NA4002, NA5002, NA6002, NA7002, and NA8002, were used. These hsRNAs were produced from identical templates via in vitro transcription as shown in Example 1, “1. Preparation of hsRNAs having two 3′-overhangs”, and a longer hsRNA has identical sequence region and additional sequence region compared to a shorter hsRNA. For example, NA1501 has 140 nt identical sequence region and 50 nt additional sequence region compared to NA1001. The dsRNA region has 106 to 806 nt in length and two 3′-overhangs have constant length, i.e., 17 nt.

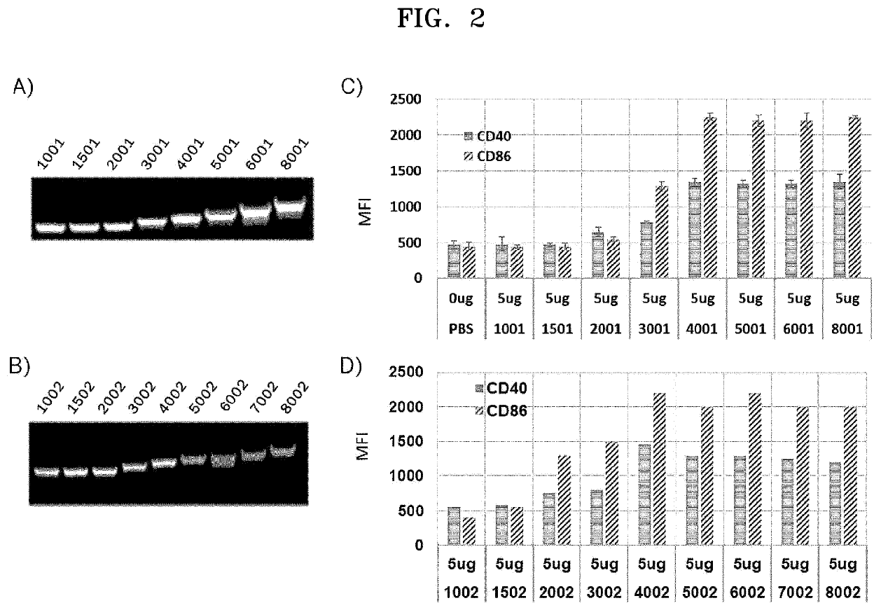

FIG. 2 shows an activity of DC according to the length of the hsRNA.

A and B of FIG. 2 are results of 1% agarose gel electrophoresis for 17 hsRNAs. NA1001, NA1501, NA2001, NA3001, NA4001, NA5001, NA6001, and NA8001 has 140 nt, 190 nt, 150 nt, 240 nt, 340 nt, 440 nt, 540 nt, 640 nt, and 840 nt in length, respectively. NA1002, NA1502, NA2002, NA3002, NA4002, NA5002, NA6002, NA7002, and NA8002, has 140 nt, 190 nt, 150 nt, 240 nt, 340 nt, 440 nt, 540 nt, 640 nt, 740 nt, and 840 nt in length, respectively.

As shown in C and D of FIG. 2, the NA2001, NA3001, NA4001, NA5001, NA6001, NA8001, NA2002, NA3002, NA4002, NA5002, NA6002, NA7002, and NA8002 increased expression of stimulatory cofactors CD40, and CD86 on the cell surface of CD11c+DC cells from spleen on 24 hours after treatment as measured by mean fluorescence intensity (MFI). MFI levels for CD40 and CD86 of the above-mentioned hsRNA injection were higher than that of the negative control, PBS injection. This indicates that the hsRNAs induce differentiation of DC maturation. The MFI was measured by flow cytometry for DCs isolated from spleen.

These data indicate that when two 3′-overhangs have 17 nt in length, respectively, and thus, the length of single-stranded RNA regions is 34 nt, the hsRNA having dsRNA region with more than 156 nt in length increased cell surface expression of CD40, and CD86 on CD11c+DC cells. The hsRNA may have dsRNA region with length 157 nt or longer, 206 nt or longer, 306 nt or longer, 406 nt or longer, 506 nt or longer, 606 nt or longer, 706 nt or longer, 806 nt or longer, 157 to 806 nt, 206 to 806 nt, 306 to 806 nt, 406 to 806 nt, 506 to 806 nt, 706 to 806 nt, 157 to 806 nt, 206 to 806 nt, 306 to 806 nt, 406 to 806 nt, 506 to 806 nt, 706 to 806 nt, 157 to 706 nt, 206 to 706 nt, 306 to 706 nt, 406 to 706 nt, 506 to 706 nt, 157 to 606 nt, 206 to 606 nt, 306 to 606 nt, 406 to 606 nt, 506 to 606 nt, 157 to 506 nt, 206 to 506 nt, 306 to 506 nt, 406 to 506 nt, 157 to 406 nt, 206 to 406 nt, 306 to 406 nt, or 366 to 866 nt.

The hsRNA may have 191 nt or longer, 240 nt or longer, 340 nt or longer, 440 nt or longer, 540 nt or longer, 640 nt or longer, 740 nt or longer, 840 nt or longer, 157 to 840 nt, 240 to 840 nt, 340 to 840 nt, 440 to 840 nt, 540 to 840 nt, 740 to 840 nt, 157 to 840 nt, 240 to 840 nt, 340 to 840 nt, 440 to 840 nt, 540 to 840 nt, 740 to 840 nt, 157 to 740 nt, 240 to 740 nt, 340 to 740 nt, 440 to 740 nt, 540 to 740 nt, 157 to 640 nt, 240 to 640 nt, 340 to 640 nt, 440 to 640 nt, 540 to 640 nt, 157 to 540 nt, 240 to 540 nt, 340 to 540 nt, 440 to 540 nt, 157 to 440 nt, 240 to 440 nt, 340 to 440 nt, or 400 to 900 nt.

### Example 3. An Antigen-Specific Antibody Response Depends on the Length of hsRNA

12 hsRNAs, i.e., R1, R2, R3, R₄, R5, R6, R7, R8, R9, R10, R11, and VP11 have an extensive length range from 353 to 1682 nt and were derived from a template DNA different from the other hsRNAs shown in Tables 1 and 2. VP11 is a hsRNA obtained by cleaving NVT with RNase T1. VP11 has a 424 bp dsRNA in the middle region flanked by 3′-overhanging UAUAG at both 3 ends. VP11 has the UAUAG sequence at both 3′-ends of the SEQ ID NO: 60.

Following the transient co-transfection into HEK 293 cells of these hsRNAs along with the IFN-β promoter-operatively linked a firefly luciferase and SV40 promoter-linked Renilla luciferase as a control, the relative level of firefly luciferase to Renilla was determined luciferase as a surrogate normalized IFN-β promoter by Firefly/Renilla Dual Luciferase® Assay (Merck cat #SCT152). The surrogate reporter level was increased proportionally to the length of the hsRNA, reaching a maximum at the range of 500 to 1066 nt. This indicates that the reporter gene expression depends on the length of the hsRNA and the nucleotide sequence of the hsRNAs has little effect to the reporter gene expression.

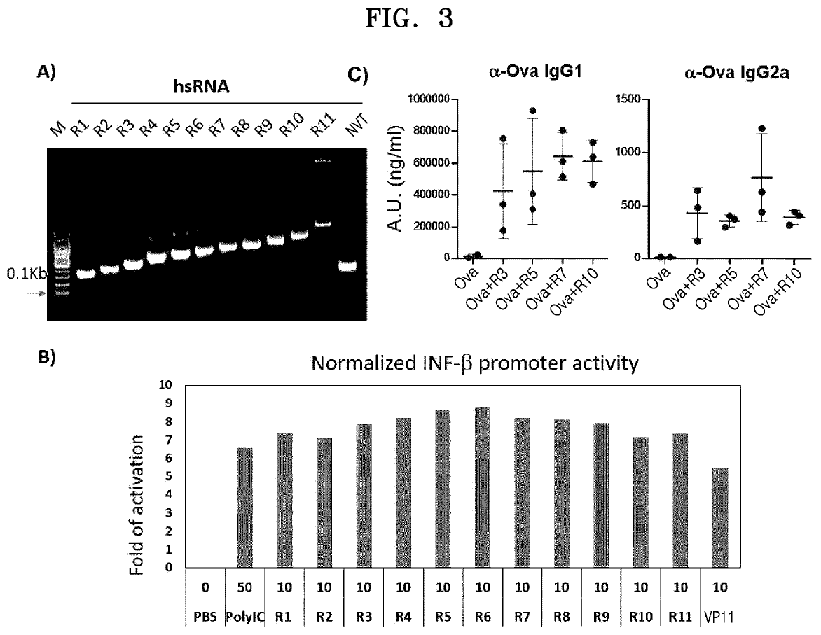

FIG. 3 shows the innate immunity activity marker and immune response result of the hsRNA.

A of FIG. 3 is a photograph of the electrophoresis in 1% agarose gel for confirming the length after preparing the hsRNA.

B of FIG. 3 shows the IFN-β increases in response to transient co-transfection of the hsRNA and a vector comprising an interferon-beta promoter and luciferase gene operatively linked to the promoter in HEK 293. As shown in B of FIG. 3, the hsRNA, i.e., R1, R2, R3, R₄, R5, R6, R7, R8, R9, R10, and R11 at 10 μg increased the IFN-β promoter activity compared to 50 μg of the positive control, PolyIC.

Several of these hsRNAs, i.e., R3, R5, R7, and R10, were mixed with a model antigen ovalbumin (OVA) in PBS, and the resultant mixture was injected into mice to test for their induction effect of anti-OVA IgG antibody in mice.

C of FIG. 3 shows that intramuscular administration of the hsRNA-adjuvanted ovalbumin in mice induced anti-OVA IgG1, i.e., Th2-polarized and IgG2a, i.e., Th1-polarized in serum. This indicates that the hsRNA enhances Th1 and Th2 cell responses in mice. The R7 hsRNA strongly enhanced the Th1 and Th2 cell response compared to R3, R5, and R10.

Consistent with the above, high levels of anti-OVA IgG1 and anti-OVA IgG2a were observed in the vicinity of R5, wherein hsRNA and dsRNA region have 698 nt and 664 nt in length, respectively.

Taken all together, the hsRNAs having 140 to 1682 nt in length, wherein dsRNA region having 106 to 1648 nt in length, exhibited excellent activation effects on innate and adaptive responses.

Therefore, the hsRNAs may have 112 to 2018 nt, for example, 140 to 1682 nt in length. The dsRNA region in the hsRNA may have 85 to 1977 nt, for example, 140 to 1682 nt in length.

### Example 4. Analysis of Physicochemical Properties of the hsRNA

The physicochemical properties of hsRNA NVT having sequence of SEQ ID NOs: 30 and 61, were measured using HPLC analysis and an agarose gel electrophoresis.

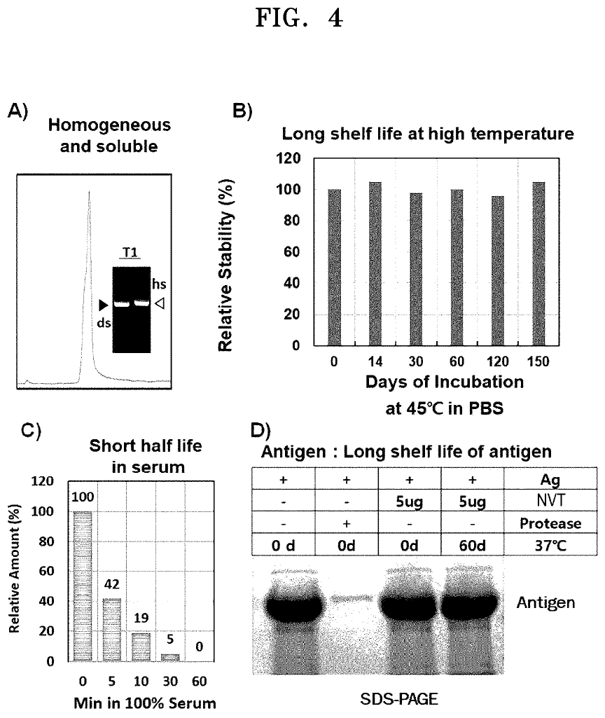

FIG. 4 shows the physicochemical properties of hsRNA NVT.

A of FIG. 4A is a photogram of 1% agarose electrophoresis, showing that the hsRNA NVT and the hsRNA NVT treated with RNase T1 have uniform length. In this experiment, the hsRNA NVT was resuspended in 1×PBS. For RP-HPLC analysis, 1 ug NVT solution in PBS was loaded to ion-pair RP-HPLC column (Waters® XBridge® OST C18 Column, 130A, 2.5 μm, 4.6×50 mm) and eluted with Acetonitrile/0.1M TEAA (25/75, v/v). The NVT solution was mixed with 1~2 unit RNase T1 and incubated for 30 min. Then the solution was subjected to HPLC reanalysis in the same way.

B of FIG. 4 shows that the hsRNA NVT treated with RNase T1 is stable at 45° C. for 150 days or longer. In this experiment, the hsRNA NVT was treated with RNase T1 as above, and the obtained solution in PBS was incubated at 45° C. for 150 days or longer. The relative stability was determined by measuring A260 for the sample obtained at the indicated day.

C of FIG. 4 shows that NVT has a half-life of shorter than 10 minutes in 100% calf serum. The serum is abundant in the RNase A and may degrade the hsRNA.

D of FIG. 4 shows that the NVT adjuvanted antigen has a longer shelf life. The antigen complex to the NVT could be stored for an extended time at room temperature. In this experiment, the ovalbumin antigen and proteinase K (MERK, cat #RPROTKSOL-RO) were used, respectively.

### Example 5. Immune Profiling Between the hsRNA and the hsRNA Treated with RNase T1

Following subcutaneous injection of (i) no RNase T1 treated-hsRNA (NVT4-T1), (ii) RNase T1-treated hsRNA (NVT4+T1) into BalB/C mice, the innate immune markers were compared in vivo after 24 and 48 hours as follows using NVT4 comprising the sequences of SEQ ID NOs: 31 and 62.

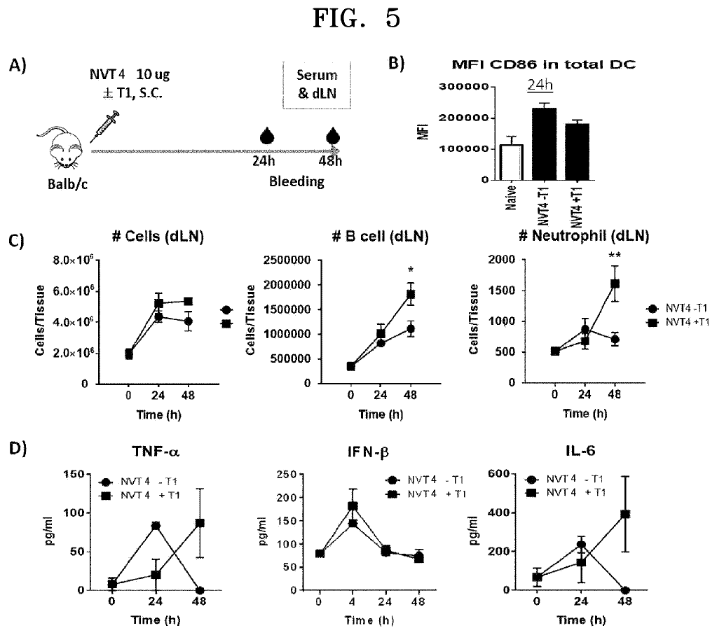

FIG. 5 shows the pattern of innate immunity activity markers after subcutaneous injection of the hsRNA in a mouse. A of FIG. 5 is a schematic immunization protocol, in order to show that the hsRNA (NVT4-T1) elicits the lower proinflammatory cytokine responses than the RNase T1 treated-hsRNA (NVT4+T1). B of FIG. 5 shows that subcutaneous injection of the RNase T1 non treated hsRNA NVT4 stimulates higher DC activation, i.e., CD86 induction, than that of the RNase treated-hsRNA NVT4 at 24 hours.

C of FIG. 5 shows immune cell profiles in the drain lymph node (dLN) after 24 hours and 48 hours of the hsRNA injection. After 48 hours, the RNase T1 treated-hsRNA NVT4 was found to have a higher immune stimulation activity than that of the hsRNA NVT4, which was not treated with RNase T1. D of FIG. 5 shows that RNase T1 treated-hsRNA NVT4 highly induces TNF-α, IFN-β, and IL-6 as compared to that of hsRNA NVT4, which was not treated with RNase T1. It suggests that hsRNA NVT4, which was treated with RNase T1, more strongly induces innate immunity.

While both NVT4+T1 and NVT4-T1 strongly induced DC activation, NVT4+T1 increased more immune cells, especially B cells and neutrophils, as compared to NVT4-T1, which was further correlated with increases in TNF-α, IFN-β, and IL-6 in proportion thereto. Taken together, NVT4+T1 induces innate immunity more strongly. NVT4-T1 elicited neutrophil, IL-6, and TNF-α, at the lower level.

### Example 6. Comparison of Th1 Polarized Immune Response Among NVT and Other Adjuvants

The OVA was resuspended in PBS with NVT, Alum, squalene (SE), IFA, PolyIC and then administered intramuscularly into mice to compare an immune response induction.

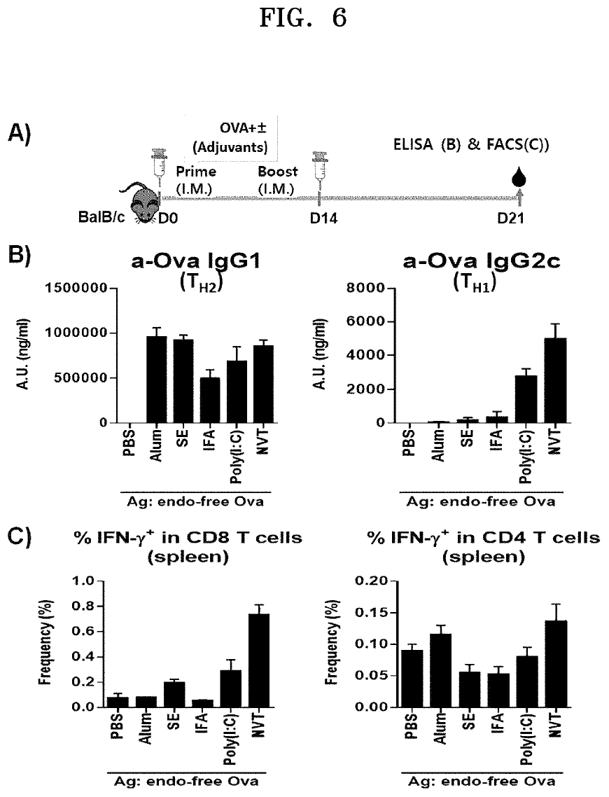

FIG. 6 shows that the NVT hsRNA-adjuvanted OVA vaccine induces a Th1 polarized immune response more strongly than other adjuvants. The NVT comprises the sequence of SEQ ID NOs: 30 and 61.

A of FIG. 6 shows an adjuvanted or non-adjuvanted vaccination intramuscularly at day 0 followed by boosting at day 14 in BalB/c mouse. The peripheral blood and splenocytes are extracted on day 21 for flow cytometry and IgG quantitation via ELISA.

B and C of FIG. 6 shows that the NVT hsRNA activated Th1 response (IgG2c) to a higher level than other adjuvants and increased the number of CD8 T cells and CD4 T cells that secrete IFN-γ in the spleen, but not the Th2 immune response (IgG1).

Though the Th2 response (IgG1) was similar, a higher Th1 response (IgG2c) resulted from NVT hsRNA-adjuvanted OVA than others. In addition, more splenic CD8 and CD4 T cells that secret IFN-γ increased in response to NVT-adjuvanted OVA vaccine formats. When administered intranasally, NVT induced more strongly mucosal IgA and mucosal IgG in the body fluids (tear, nasal cavity, and drainage) and serum than other adjuvants (data not shown).

### Example 7. Innate and Antigen-Specific Antibody Responses Between hsRNA and hsRNA Plus Additional Adjuvant

Two formulations of (i) NVT; the hsRNA comprising the sequence of SEQ ID NOs: 30 and 61, (ii) NVT II; NVT+SE (squalene emulsion) was prepared. NVT was prepared by solubilizing the NVT hsRNA in PBS, and NVT II was prepared by mixing the 50% hsRNA NVT, 4.3% squalene, 0.5% Span 0.5% Tween® 80 in PBS.

Following intramuscular injection of NVT and NVT II into BalB/C mice, DC activity was measured by flow cytometry. DC stimulation in the inguinal lymph node (ILN) was higher in NVT II than in NVT.

In addition, model vaccines, including (i) OVA alone, (ii) OVA+NVT, (iii) OVA+NVT II were prepared and tested in the same mice for the anti-OVA IgG1, indicating Th2 response, and IgG2a, indicating Th1 response.

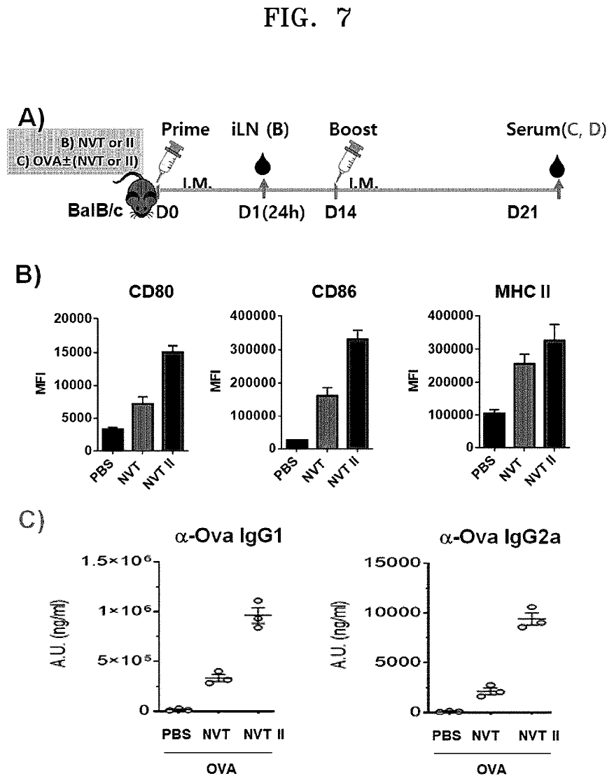

FIG. 7 shows the comparison of NVT and NVT II in an innate immune and adaptive immunity induction. A of FIG. 7 is a dosing regimen for immunization with the hsRNA.

B of FIG. 7 shows that NVT II more strongly induces the DC activity in inguinal lymph node (iLN) over NVT and PBS (phosphate-buffered saline) 24 hours after injection.

C of FIG. 7 shows the level of binding antibody to the ovalbumin antigen when injected with PBS, NVT, and NVT II.

As a result, OVA+NVT induced a stronger antibody response by about 100 times than OVA alone. The OVA+NVT II induced a stronger antibody response by 2 times or more than OVA+NVT.

### Example 8. Homogeneity in Structure and Length of the hsRNA

The hsRNA has a dsRNA region and two ssRNA overhangs at both 3′ ends. Both dsRNA region and overhangs have a non-homopolymeric, i.e., heteropolymeric sequence and can be defined as an exact length, preventing homopolymer-mediated chain slippage and extension.

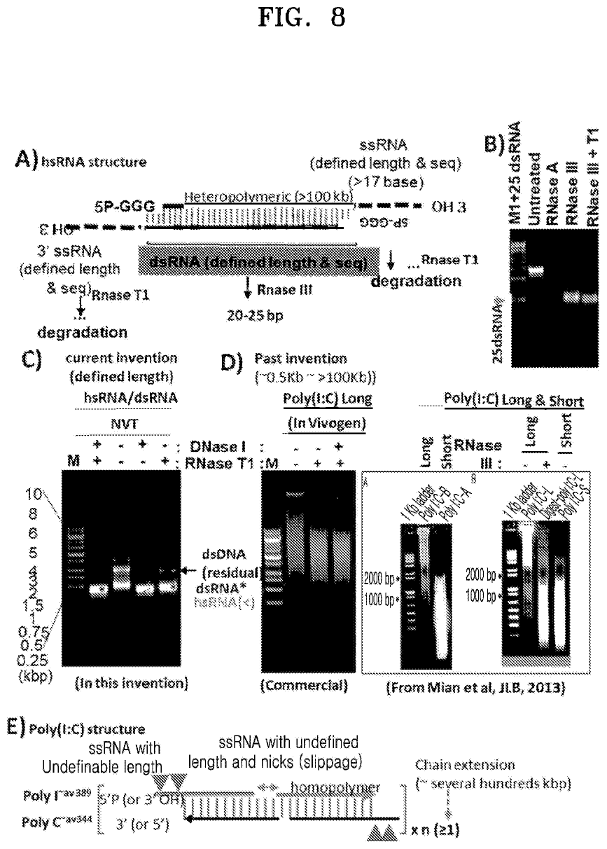

FIG. 8 shows the structure of the hsRNA. Heteropolymeric dsRNA region is flanked by each ssRNA overhangs at the 3′ ends.

A of FIG. 8 is a schematic diagram of the hsRNA. The heteropolymeric dsRNA region of the hsRNA has a certain length and any particular sequence with complete complementarity between single strands. The two ssRNA overhangs to both 3′ ends of the dsRNA can be any certain length with no complementarity. The two overhangs may be capable of acting as a potential TLR7/8 ligand. The dsRNA region may be an artificial sequence or may be a naturally occurring sequence. The dsRNA region may not encode a protein. Upon RNase III treatment, the dsRNA region is cleaved into pieces of dsRNA of 20 to 25 nt on average. However, RNase T1, which does not act on dsRNA but acts only on ssRNA overhang. The RNase 1 may specifically cleave single-stranded RNA at G residues. It may cleave the phosphodiester bond between the 3′-guanylic residue and the 5′-OH of adjacent nucleotides. The reaction products may be 3′-GMP and oligonucleotides with a terminal 3′-GMP. The RNase I may reduce the length of ssRNA overhangs. The present invention provides the advantage in which hsRNA having a length can be mass-produced in vitro using a T7 RNA polymerase at a similar molar ratio of two complementary ssRNAs derived from both strands of DNA. The obtained hsRNA may have more than 100 nt in length.

In the present invention, the ssRNA overhang may have a specific length and sequence. For example, the overhang may have a sequence comprising a large number of GU sequences, a TLR7 ligand sequence. The overhang may be capable of acting as one particular TLR7/8 ligand. In addition, the overhang may have a function to prevent the end of the dsRNA region from being degraded.

Bidirectional transcription from the one template DNA generates two complementary two ssRNAs with almost the same molar ratio. During the IVT, two strands subsequently undergo spontaneous annealing to forms hsRNA with perfect dsRNA region and two ssRNA overhang in both 3′ ends. Of particular importance, the two ssRNA overhangs are designed not to be complementary to each other to prevent base pairing or chain extension.

B of FIG. 8 shows a result of digesting hsRNA with RNase A, RNase III, and RNase III+RNase T1. RNase A can degrade both dsRNA and ssRNA degraded all RNAs. When treated with RNase Ill that can cleave dsRNA into 20 to 25 nt in length, the band of segments with a range of about 25 nt appeared most. The RNase III does not cleave ssRNA overhangs, which are 51 nt and 58 nt, respectively, of the hsRNA NVT4. So ssRNA overhangs remain undegraded, which overlapped around 25 nt. When treated with RNase III and RNase T1 at the same time, the ssRNA segments with 51 nt and 58 nt were completely degraded and disappeared, and only a single band of 25 nt appeared clearly.

C of FIG. 8 shows that NVT comprising the sequence of SEQ ID NOs: 30 and 61 and having 533 nt in length and dsRNA region with 424 nt in length was isolated in agarose gel before and after treatment with DNase I and RNase T1, and then a constant result appeared with a specific length. After IVT, residual DNA templates are removed with the treatment of DNase I, and then the only hsRNA remains. When the ssRNA overhang was present in hsRNA, its length was reduced by RNase T1. If there are one or more nicks within the dsRNA region, RNase T1 will cleave the nick site, resulting in two or more dsRNA segments. In contrast, only one band in agarose gel demonstrates that there are no nicks in the dsRNA sites and the ssRNA overhangs.

D of FIG. 8 shows that PolyIC, a commercially available positive control, has a heterogeneity due to the extreme length diversity which arises from the preparation stage, which cannot be overcome even after finishing the fractionation after preparation (Mian M F et al., (2013) J Leukoc Biol 94 (5): 1025-36).

E of FIG. 8 shows the basic structure of PolyIC, a commercially available positive control, which can be represented only as an average length, not a specific length. For example, homopolymeric Poly(I) of about 389 bases on average and Poly(C) of 344 bases on average are synthesized respectively. They are allowed to complementarily anneal to each other, and then dsRNA is prepared. However, because chain slippage, i.e., random site base pairing, occurs in which complementary associations occur at unpredictable, non-specifiable sites, the formation of multiple nicks with a variable gap in unpredictable locations is unavoidable. Besides, the random site base-pairing should create the unspecifiable base stretch of Poly(I) and poly(C) tails at both places regardless of 5′ or 3′ ends, to which complementary strands associates again, leading to chain extension. Therefore the length is extended to several hundred kilobases or more (Mian M F et al., (2013) J Leukoc Biol 94 (5): 1025-36). In addition, since PNPase is used in the preparation process of PolyIC, a phosphate group (P) is always attached to 5′ end, and thus the yield is low.

In contrast, the hsRNA can be designed to form dsRNA flanked by overhangs only at both 3′ ends having a defined length with no complementary sequence each other that prevents chain extension, which is in contrast to PolyIC and its derivatives.

As another specific example, any particular single strand containing polyuridine (U) base can be added to both sides of the dsRNA region. In addition, a single strand can be designed to have stability by adding a specific sequence to form an internal dsRNA such as a hairpin structure or a palindrome ring structure or to act as a TLR7-like ligand.

In contrast, PolyIC, a competitive drug, has extreme length diversity and cannot be prepared at a constant length. Instead, it is artificial homopolymeric dsRNA with unspecifiable nicks insides and has no ssRNA tail in specified sites such as the 3′ end of dsRNA. The length diversity of PolyIC is caused by slippage within the chain and chain extension. As a result, multiple nicks in unspecifiable sites in the dsRNA site are formed, which are cleaved by RNase T1 into various lengths (D of FIG. 8). In addition, the number of the upper and lower strands forming dsRNA cannot be specified due to uncontrolled chain extension in PolyIC. Thus PolyIC is easily digested by RNase T1. The extreme heterogeneity of PolyIC was published in one of the original articles (Mian M F et al., (2013) J Leukoc Biol 94 (5): 1025-36).

RNase Ill digestion of PolyIC-L (long species) gave rise to small species similar to PolyIC-S (short species) (D of FIG. 8). This extreme heterogeneity was partially improved by reducing chain extension through complementary binding a short PolyC (P100 of 100 nts on average) to a long Polyl (P400 of 400 nts on average). Nonetheless, the length varies in a vastly diverse range from 100 nt to 100 knt.

The RNA has an essential difference from the existing invention in the following matters.

In contrast to PolyIC, the RNA 1) has a high homogeneity in length, is a non-homopolymer of sequence, and does not have nicks in dsRNA due to complete complementarity, and 2) may act as a TLR3 ligand and a TLR7-like ligand.

As compared to siRNA, the hsRNA 1) has an essential difference in the preparation process as compared to the synthesized siRNA, 2) does not have a function of target-suppressing a specific gene possessed by siRNA, and 3) can act as a TLR3 ligand exhibiting sufficient activity because the length of the hsRNA is 45 nt or more, whereas the range of siRNA is short as 21 to 25 nt, so siRNA cannot stimulate TLR3, and 4) the ssRNA of the present invention has a specific sequence of a particular length or more extended, for example, 1 nt or more, or 17 nt or more, and acts as TLR7/8-like ligand, whereas a very short 3′-overhang at the end of the siRNA corresponds to a simple residual sequence without function.

### Example 9. Immune Response to dsRNA Adjuvanted-Whole Inactivated Influenza Vaccine (iPR8) Via Intranasal Administration

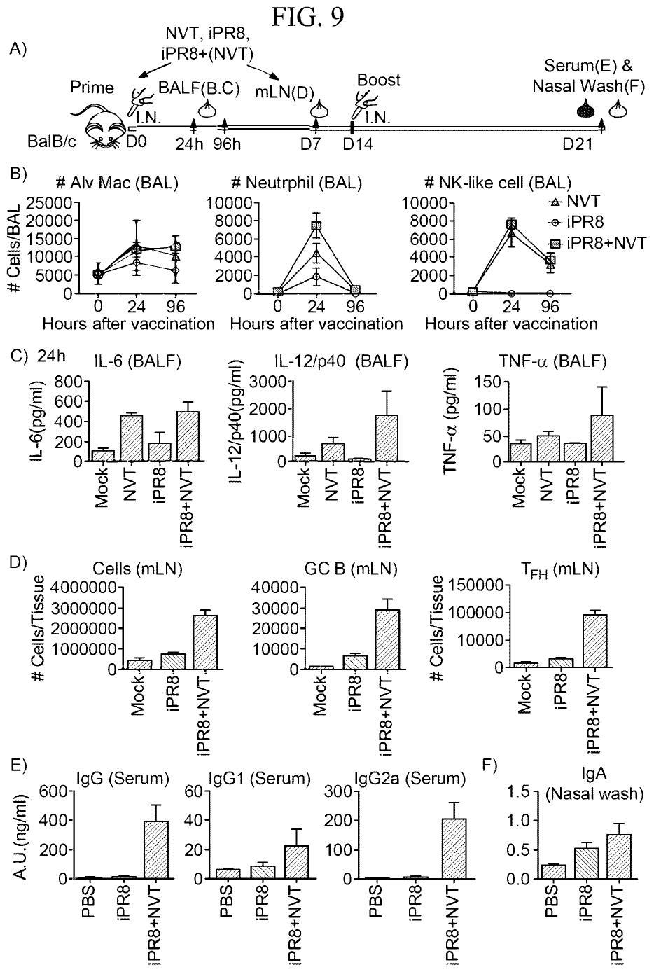

When NVT was administered together with iPR8 to BalB/c mice, the number of mDC and rDC cells increased in the mediastinal lymph nodes after 24 hours, and DC (CD80, CD86) was activated and then declined to a baseline level after 96 hours (data not shown). In addition, total cells, alveolar macrophages, neutrophils, and natural killer cells in bronchoalveolar lavage fluid were increased (B of FIG. 9), which was proportional to the increase in IL-6, IL-12, and TNF-α, wherein NVT alone also showed a positive effect on the rise of those proinflammatory cytokines (C of FIG. 9). This increase in innate immunity marker was accompanied by an increase in germinal center B (GCB) cells, follicular helper T cells (TFH), and IgG and IgA in serum and nasal lavage fluid (D and E of FIG. 9).

The NVT adjuvant alone also had a protective effect against a lethal live viral infection in the absence of an antigen. When the NVT was intranasally administered several times to the mice, the lifespan was extended by 4 days or more on average, and the mortality rate related to virus infection (viremia) was reduced by about 20% (data not shown). Again, the NVT adjuvant alone without an antigen exhibited a robust immune enhancement action.

FIG. 9 shows the immune response after intranasal administration of the whole inactivated influenza vaccine (iPR8) adjuvanted with (iPR8+NVT) or without (iPR8) the NVT or NVT alone.

A of FIG. 9 is a schematic diagram of the test method.

B of FIG. 9 shows profiles of alveolar macrophages, neutrophils, and natural killer cells in bronchoalveolar lavage fluid from n NVT, iPR8, and iPR8+NVT-treated group mice.

C of FIG. 9 shows a marked increase in IL-6, IL-12, and TNF-α after 24 hours.

D of FIG. 9 shows an increase in germinal center B (GCB) cells and follicular helper T cells (TFH) in mediastinal lymph node (mLN) on day 7.

E of FIG. 9 shows that serum anti-iPR8 IgG increases after 21 days in the iPR8+NVT group.

F of FIG. 9 shows an increase in IgA in nasal lavage fluid on day 21.

### Example 10. Effect of the hsRNA on Dose Sparing of Commercial Influenza Split Vaccine

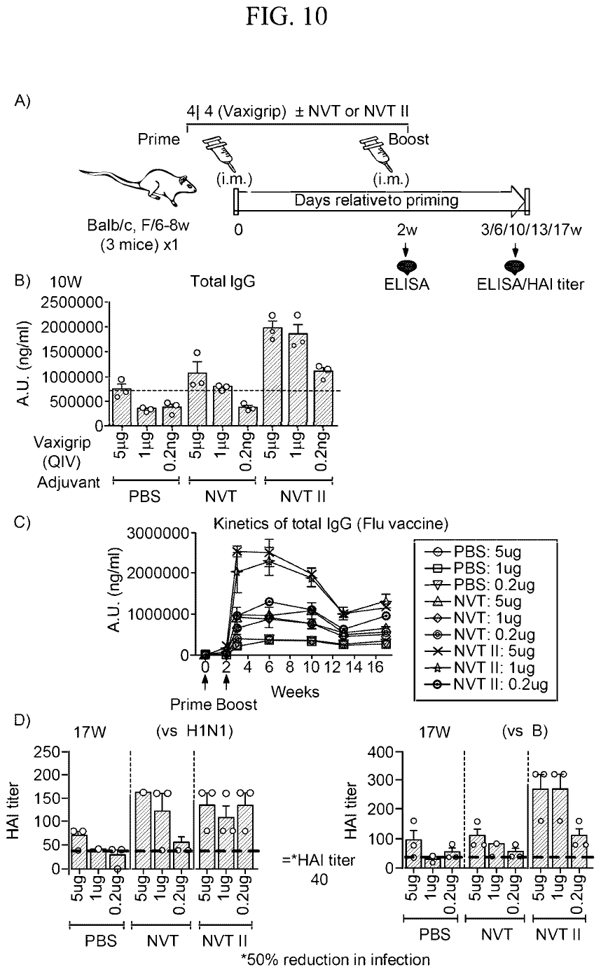

The adjuvant role in the commercial vaccine antigen was tested. Tested vaccines include (i) tetravalent inactivated influenza vaccine (4IIV, Vaxigrip®, Sanofi Pasteur), (ii) 4IIV+NVT, (iii) 4IIV+NVT II. NVT II refers to (NVT+SE) formulation. Following intramuscular priming and boosting apart 2-weeks intervals in female Balb/c mice, IgG level and hemagglutination inhibition (HAI) titer were determined over the 18 weeks of post priming (A of FIG. 10).

FIG. 10 relates to an effect of the hsRNA adjuvant of this invention on an efficacy and antigen dose saving of a commercial influenza vaccine.

A of FIG. 10 is a schematic diagram of the test method.

B of FIG. 10 shows that NVT or NVT II adjuvants saved the amount of antigen required to induce IgG to about ⅕ and about 1/25 or less, respectively. Vaxigrip®, which is 2018/2019 seasonal tetravalent influenza vaccine 4IIV, was used as a model antigen

C of FIG. 10 shows that the higher amount of total IgG in NVTI and NVT II groups was maintained until 17 weeks after vaccination.

D of FIG. 10 shows that the hemagglutination inhibition (HAI) titer ≥40 was maintained at least over 40 until week 17 in proportion to the amount of neutralizing antibodies. HAI ≥40 is a correlate of protection (COP) in which at least 50% of vaccinated individuals are protected.

As a result, the antigen dose could be reduced to about ⅕ level in NVT− and about 1/25 in NVT II adjuvanted groups as compared to non-adjuvanted antigen alone (B of FIG. 10). This effect lasted for at least 17 weeks after injection (C of FIG. 10). NVT adjuvant in this invention could spare an antigen dose; the amount of vaccine antigen required to secure the needed immunity can be considerably reduced.

The increase in binding antibody was closely related to the rise in HAI titer of 40 or more, which is a correlate of protection (COP) against the influenza virus, likely capable of providing sufficient protective immunity to 50% of challenged or infected individuals. Though both adjuvants considerably increased HAI titer, NVT II especially elicited the HAI titer by 3 to 5-fold to both influenza A virus (IAV subtype H1N1) and influenza B virus (IBV) (D of FIG. 10).

### Example 11. Dose Sparing Effect by hsRNA on Commercial Meningitis Vaccine

NVT was used as an adjuvant while administering the Menactra® vaccine to mice. The Menactra® vaccine is a CRM197-conjugate vaccine against Neisseria meningitidis groups A, C, Y, and W-135. Specifically, vaccine antigens were covalently linked to the diphtheria toxin-derived carrier protein CRM197.

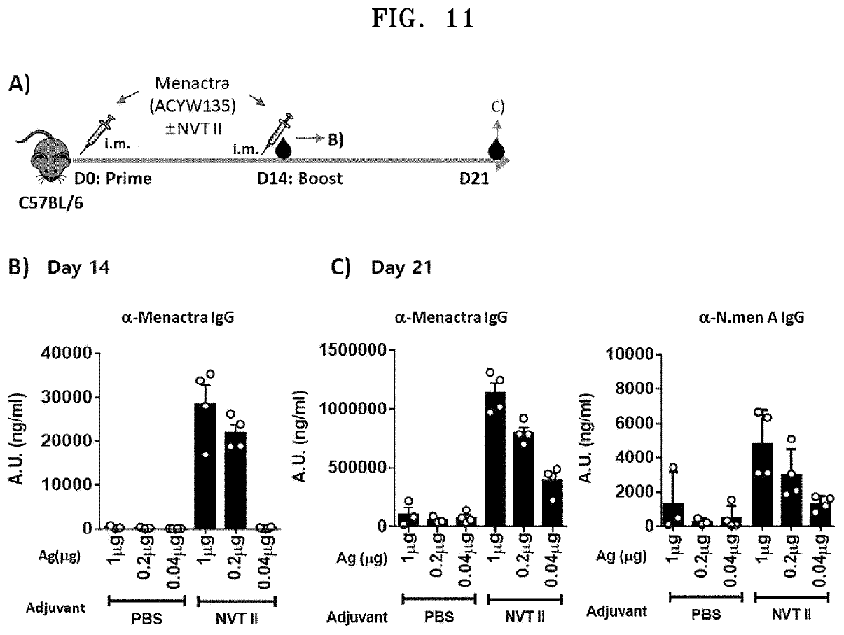

FIG. 11 shows the induction in antibody IgG against a meningococcus vaccine (Menactra®, Sanofi) adjuvanted with the NVT II. FIG. 11A is a schematic diagram of the test method.

B and C of FIG. 11 show antibody increase against the total conjugated antigen (Menactra®) and A type of antigen at 14 and 21 days, respectively, after priming. As shown in FIGS. 11B and 11C, NVT II-adjuvanted Menactra® vaccine strongly induced Menactra® specific antibody by 5 times or more.

### Example 12. Immunogenicity Restoration Effect of NVT II

The influenza vaccine (2018/2019 QIV Vaxigrip®) was incubated at 37° C. for 5 weeks to abolish immunogenicity, followed by adjuvanted with NVT II and injected intramuscularly. The IgG ELISA and HAI assay were performed at indicated time points until 18 weeks post-priming.

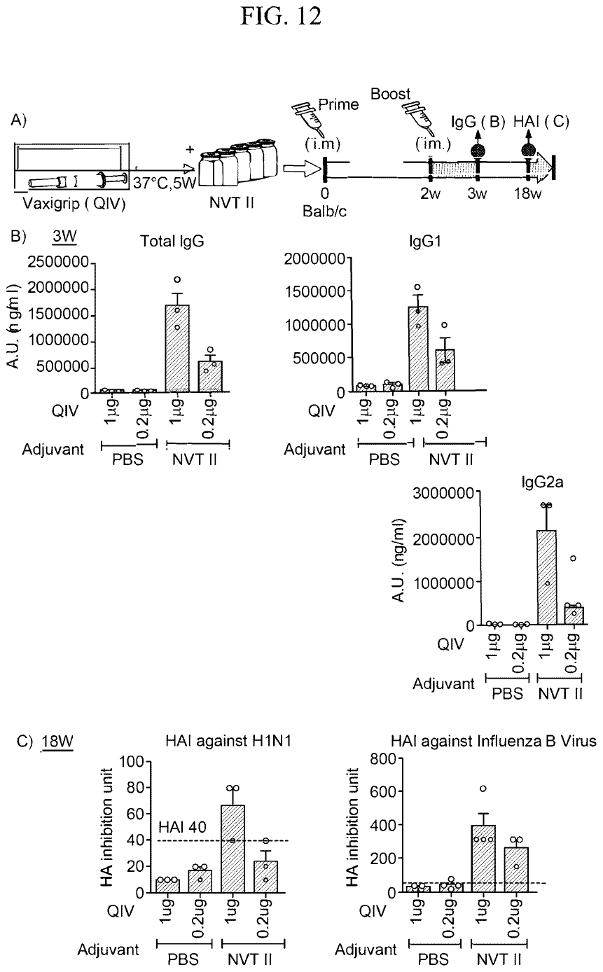

FIG. 12 shows an effect of the NVT II adjuvant on immunogenicity restoration. A of FIG. 12 is a schematic diagram of the test method. Vaxigrip® was incubated at 37° C. for 5 weeks and adjuvanted with NVT II or PBS, and the vaccine was injected into mice via intramuscular injection according to the injection schedule as shown in A of FIG. 12. B of FIG. 12 shows that NVT II restored the immunogenicity of Vaxigrip® as assessed by the IgG antibody level. Note Vaxigrip® alone completely lost the immunogenicity. C of FIG. 12 shows that the NVT II-mediated IgG level was correlated with an increase in the HAI titer ≥50 against IAV H1N1 and IBV at week 18.

As a result, as shown in B of FIG. 12, the binding antibody was markedly increased, and the immunogenicity appeared to be restored. In addition, there was an antigen-saving effect by at least 10 times. As shown in C of FIG. 12, the binding antibody increase correlates with an increase in neutralizing antibodies as the HAI titer against H1N1 and IBV increased to 40 or more at week 18.

In another experiment, when Vaxigrip® was pre-mixed with NVT II and left at 37° C. for 5 weeks, antigenicity was fully maintained, and HAI titer was increased to 40 or more (data not shown).

### Example 13. Anti-Tumor Effect of hsRNA on Melanoma Growth In Vivo

Following subcutaneous implantation of B16F10-OVA melanoma cells to 7-week-old female C57BL/6 mice, the NVT in PBS was administered intratumorally or intramuscularly three times at an interval of 2 days from day 6.

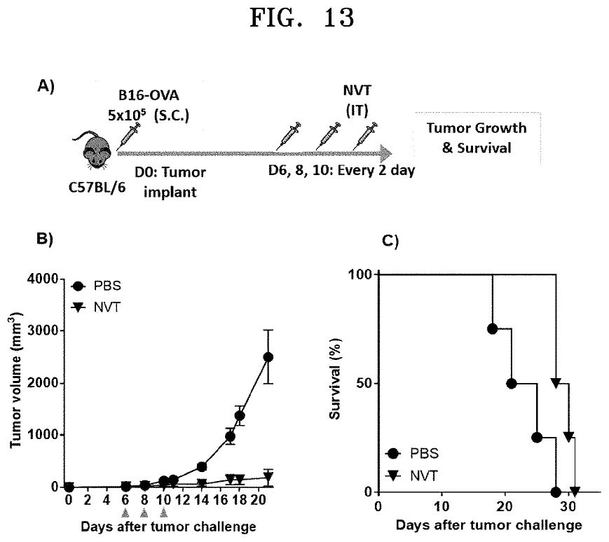

FIG. 13 shows an effect of the hsRNA alone on the arrest of melanoma growth. A of FIG. 13 is a schematic diagram of the test method. On 6 days after B16F10-OVA melanoma cell implantation to 7-week-old female C57BL/6 mice, the NVT was administered intratumoral route on days 6, 8, and 10, which was followed by measurement of the tumor mass and mice survival. B of FIG. 13 shows that the hsRNA NVT significantly arrests melanoma growth. C of FIG. 13 shows that the hsRNA NVT prolonged the survival rate of the mice.

As shown in B and C of FIG. 13, tumor growth was retarded in the mice administered with the NVT, and mice survived longer as compared to the control.

### Example 14. Inhibitory Effect of the hsRNA on the Growth of Colorectal Cancer and Lung Cancer In Vivo

Following subcutaneous implantation of CT26 colorectal cancer and LL/2 lung cancer to 7-week-old female C57BL/6 mice, NVT in PBS alone was administered subcutaneously, intratumorally or intramuscularly once every two days over a total of 8 times.

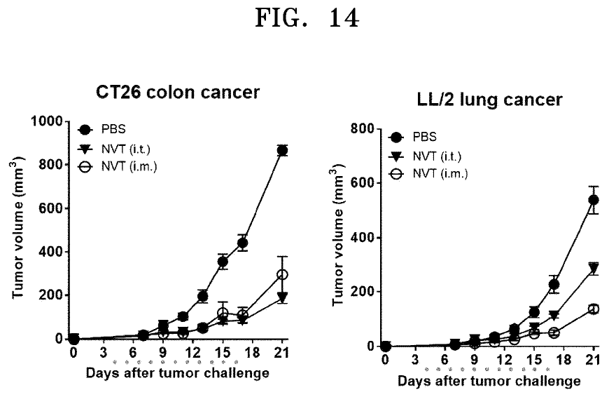

FIG. 14 shows an effect of the hsRNA NVT alone on the growth arrest of colorectal cancer and lung cancer. As shown in FIG. 14, the growth of colorectal and lung cancer in mice was retarded by the NVT treatment.

### Example 15. Inhibitory Effect of the hsRNA Alone on the Growth and Lung Metastasis of Triple-Negative Breast Cancer (TNBC) In Vivo

Following subcutaneous implantation of a 4T1 triple-negative breast cancer (TNBC) cell line to mammary adipose tissue of 7-week-old female C57BL/6 mice, the hsRNA NVT in PBS alone was intratumorally administered once every two days from day 8 over a total of 9 times.

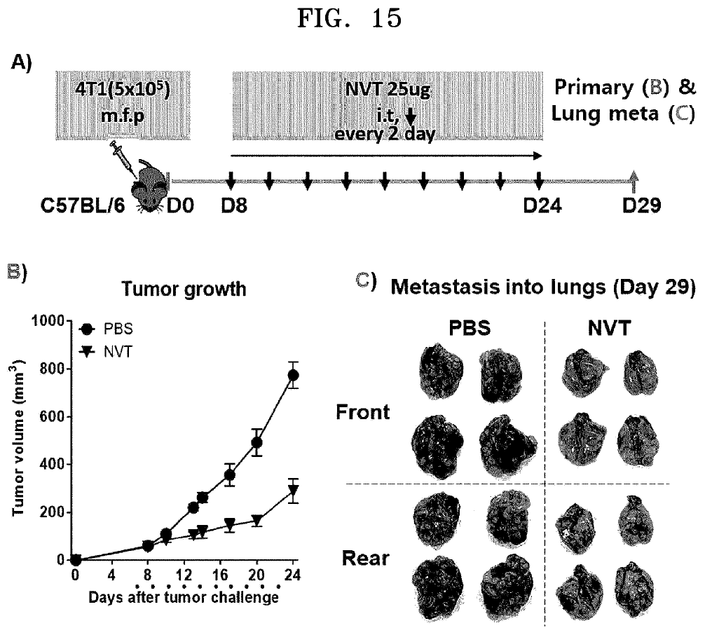

FIG. 15 shows an inhibitory effect of the hsRNA in PBS alone on the growth arrest and metastasis of triple-negative breast cancer (TNBC). A of FIG. 15 is a schematic diagram of the test method. A triple-negative breast cancer (TNBC) cells 4T1 was subcutaneously implanted to mammary adipose tissue of 7-week-old female C57BL/6 mice. The NVT hsRNA was intratumorally administered alone once every two days from day 8 over a total of 9 times, which was followed by measurement of tumor masses on indicated days. B of FIG. 15 shows that the NVT arrested the growth of the primary breast by 60%. C of FIG. 15 shows that NVT suppressed metastasis of primary breast cancer to the lung.

As shown in B and C of FIG. 15, primary breast cancer was retarded by 60%, and this growth arrest was consistent with a marked reduction in remote metastasis into the lung.

### Example 16. Anti-Metastasis to the Liver of the hsRNA Alone or hsRNA-Complexed TAA Vaccine In Vivo

The vaccines (i) PBS, (ii) OVA, (iii) NVT, (iv) OVA+NVT were pre-administered intraperitoneally on day 0. On the third day, B16F10-OVA melanoma was intrasplenically implanted into 7-week-old female C57BL/6 mice. In the following 3 days, and the same vaccines were administered intraperitoneally, and then the degree of metastasis from the spleen to the liver was investigated.

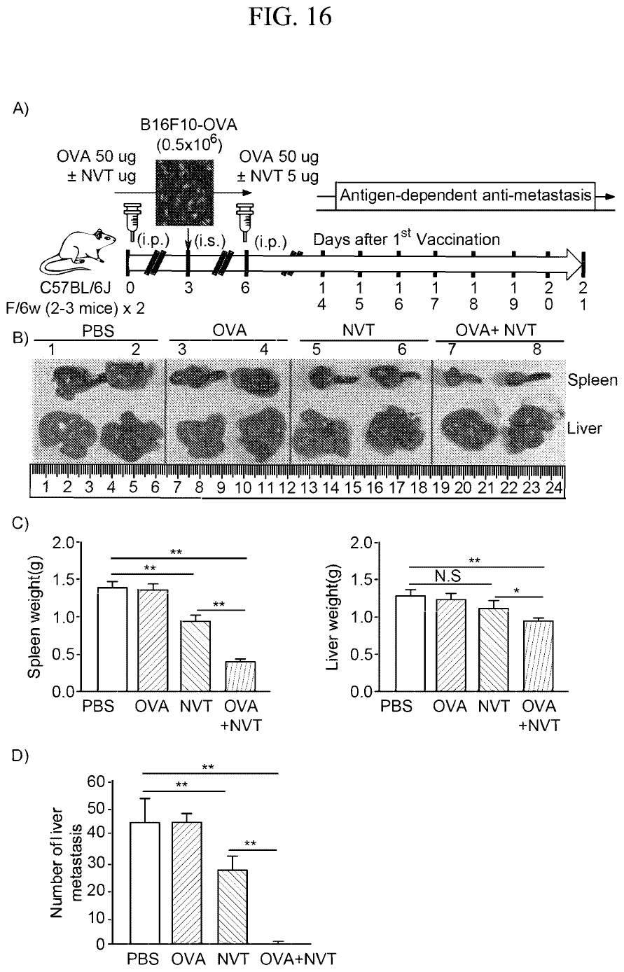

FIG. 16 shows an inhibitory effect of NVT-adjuvanted OVA cancer vaccine administered intraperitoneally on melanoma cell metastasis. A of FIG. 16 is a schematic diagram of the test method. B of FIG. 16 shows the morphology of the spleen and liver on day 18, where the size and metastatic nodules in the liver can be clearly seen.

C of FIG. 16 shows an average weight of the spleen and liver on day 18.

D of FIG. 16 shows the number of metastasized tumor nodules to the liver. The metastasis to the liver was markedly suppressed in the NVT alone, or NVT+OVA treated group.

As shown in B and C of FIG. 16, metastasis in the NVT− or OVA+NVT treated groups was lower than in the non-treated or OVA alone-treated group. As shown in D of FIG. 16, the metastasis to the liver on day 18 was lower by about 50% in NVT alone and by 90% or more in the OVA+NVT group. As for survival, whereas all mice died within 18 days, starting from day 14 in all other groups, mice in the OVA+NVT-treated group died within 28 days, starting from day 24.

### Example 17. Inhibitory Effect of the hsRNA-Adjuvanted OX40 Ab Complex on Primary and Remote Melanoma Growth

Following subcutaneous implantation of B16F10-OVA melanoma cells to the left flank of 7-week-old female C57BL/6 mice, the cancer vaccines (i) PBS, (ii) PD-1 Ab, (iii) IR cells, which are irradiated cancer cells, and (iv) IR cells+NVT+OX40 Ab was administered, respectively intratumorally into the primary cancer tissue once every two days four times (on day 6, 8, 10, and 12) after tumor implantation. The growth of the cancer was measured until day 22.

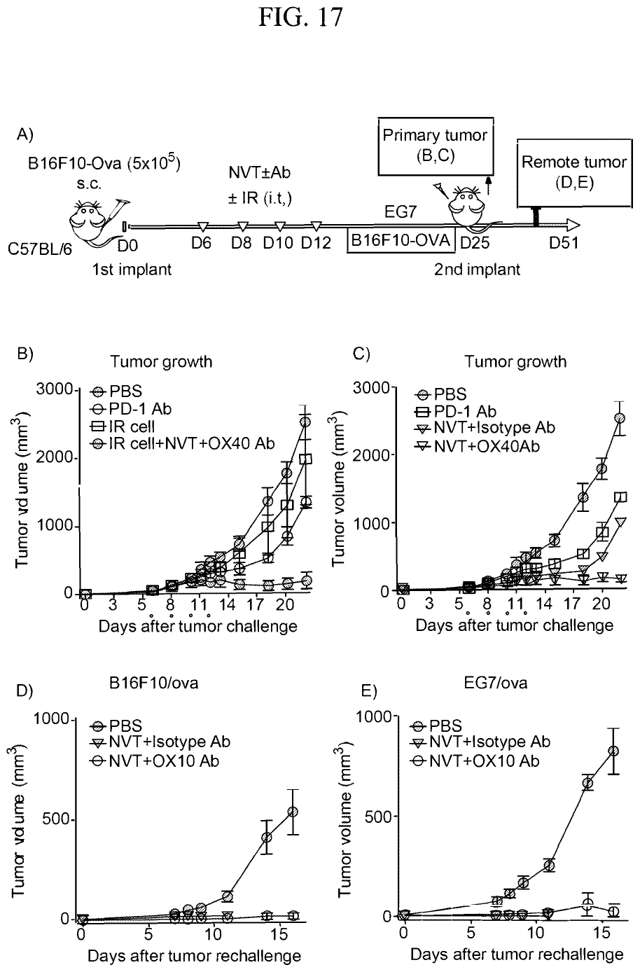

FIG. 17 shows an inhibitory effect of a cancer vaccine comprising the hsRNA NVT and an OX40 antibody on primary and remote cancer growth. A of FIG. 17 is a schematic diagram of the test method. A melanoma cell line B16F10 OVA was subcutaneously implanted to the left flank of 7-week-old female C57BL/6 mice. On day 6 post-implantation, the indicated regimens were (i.e., IR+NVT+OX40 Ab) were administered 4 times once every 2 days (primary cancer). On day 15, the same the mice received B16F10-OVA or EG7/OVA cells on the right flank (remote cancer), which was followed by measurement of tumor masses on indicated days.

B of FIG. 17 shows that irradiated cancer (IR) cell+NVT+OX40 Ab composition considerably inhibited the growth of primary cancer. C of FIG. 17 shows that NVT+OX40 Ab without IR cell fully inhibited the primary melanoma growth. D and E of FIG. 17 show that only NVT+OX40 Ab fully inhibited the remote cancer B16FO-OVA identical to the primary tumor and heterologous remote cancer EG7/OVA.

As shown in B of FIG. 17, whereas primary cancer growth was suppressed by about 50% and 20% only in the PD-1 antibody and IR cell, respectively, 90% growth arrest was observed in the IR+NVT+OX40 Ab group.

As shown in C of FIG. 17, NVT+OX40 Ab without IR cells suppressed the growth of primary cancer almost to the same extent to the above, non-necessitating the TAA in the vaccine composition.

On day 15, two cancers (B16F10/OVA) and T cell lymphoma (EG7/OVA) of allogeneic origin were implanted to the second site on the right flank on the opposite side from the first implantation. The growth was measured for the following 16 days without further administering therapeutic vaccines.

As a result, all mice of NVT+OX40 Ab injection resulted in almost no growth in the second site cancer. In contrast, all mice of the NVT+ isotype Ab group died before day 11. Therefore, the size of the mass was not shown in the figure (D and E of FIG. 17).

In summary, the growth of primary melanoma was inhibited by 90% or more with NVT+OX40 Ab regardless of IR. Besides, remote cancers implanted in the other site were also arrested by NVT+OX40 Ab.

### Example 18. Inhibitory Effect of the hsRNA-Adjuvanted PD-1 Antibody or OX40 Complex on Primary and Remote Melanoma

Following subcutaneous implantation of B16F10-OVA melanoma cells to the left and right flanks of 7-week-old female C57BL/6 mice, the cancer vaccines (i) PBS, (ii) NVT, (iii) NVT+OX40 antibody (OX40 Ab) (clone OX-86, Rat IgG1, InvivoGen), and (iv) NVT+PD-1 antibody (PD-1 Ab) (clone RMP1-14, Rat IgG2a/A, InvivoGen) were administered intratumorally into the left primary cancer tissue once every two days four times (on day 6, 8, 10, and 12) after tumor implantation. The growth of cancer in the left flank side (vaccinated tumor) and the right side (remote tumor) were measured until day 20.

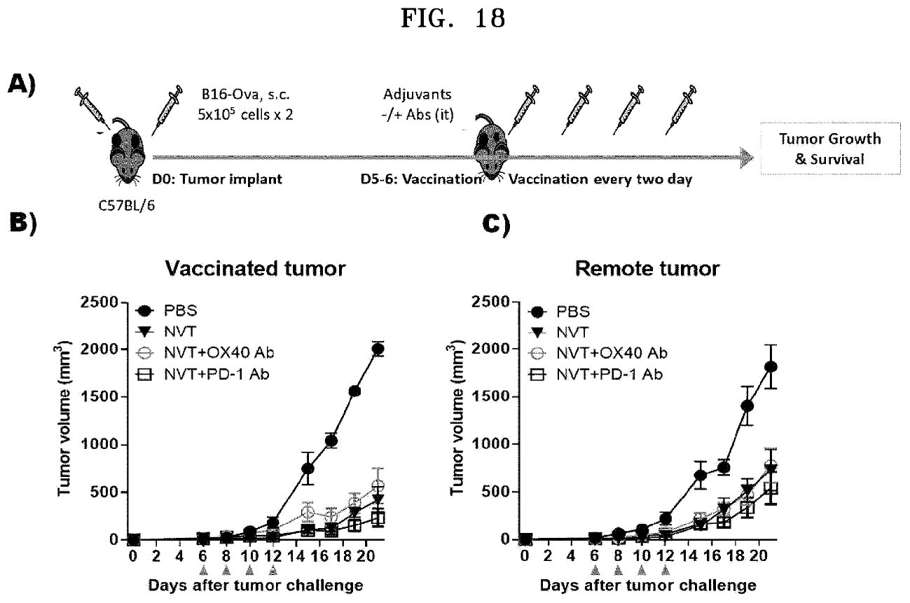

FIG. 18 shows the inhibitory effect of the hsRNA NVT in combination with the ant-PD-1 antibody or OX40 antibody on melanoma growth.

A of FIG. 18 is a schematic diagram of the test method. B of FIG. 18 shows that the tumor inhibitory effect of the NVT+PD-1 composition is generally similar to that of the NVT+OX40 Ab composition.

C of FIG. 18 shows that the two compositions have similar inhibitory effects on the tumors of remote cancer.

As a result, the inhibitory effect NVT+PD-1 Ab on primary cancer was similar to or better than that of NVT+OX40 Ab. The same results were observed in the nonvaccinated remote tumors.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-18. (canceled)

19. A hetero-structured RNA (hsRNA) comprising a heteropolymeric double-stranded RNA (dsRNA) region having a first strand and a second strand and single-stranded RNA (ssRNA) regions, wherein the ssRNA regions are positioned at the 3′-end of the first strand and the 3′-end of the second strand,
wherein the dsRNA acts as a TLR3 ligand and has a base length of 406 to 806 bp, and wherein the ssRNA acts as a TLR7-like ligand or a TLR8-like ligand and has a base length of 75 bp or less.

20. The hsRNA of claim 19, wherein the hsRNA is a endoribonuclease cleaved product obtained by endoribonuclease cleaving a hsRNA comprising a heteropolymeric double-stranded RNA region having a first strand and a second strand and single-stranded RNA regions, wherein each of the ssRNA region is positioned at the 3′-end of the first strand and the 3′-end of the second strand.

21. The hsRNA of claim 20, wherein the endoribonuclease is RNase T1.

22. The hsRNA of claim 19, wherein the ssRNA region has the UAUAG sequence at the 3′-end of the first strand and the second strand.

23. The hsRNA of claim 19, having the following nucleotide sequence set or endoribonuclease cleaved product thereof, wherein one nucleotide sequence of the set base pairs with the other nucleotide sequence of the set to form a double-stranded RNA with two 3′-overhangs:
SEQ ID NOs: 1 and 32, SEQ ID NOs: 2 and 33, SEQ ID NOs: 3 and 34, SEQ ID NOs: 4 and 35, SEQ ID NOs: 5 and 36, SEQ ID NOs: 6 and 37, SEQ ID NOs: 7 and 38, SEQ ID NOs: 8 and 39, SEQ ID NOs: 9 and 40, SEQ ID NOs: 10 and 41, SEQ ID NOs: 11 and 42, SEQ ID NOs: 12 and 43, SEQ ID NOs: 13 and 44, SEQ ID NOs: 14 and 45, SEQ ID NOs: 15 and 46, SEQ ID NOs: 16 and 47, SEQ ID NOs: 17 and 48, SEQ ID NOs: 18 and 49, SEQ ID NOs: 19 and 50, SEQ ID NOs: 20 and 51, SEQ ID NOs: 21 and 52, SEQ ID NOs: 22 and 53, SEQ ID NOs: 23 and 54, SEQ ID NOs: 24 and 55, SEQ ID NOs: 25 and 56, SEQ ID NOs: 26 and 57, SEQ ID NOs: 27 and 58, SEQ ID NOs: 28 and 59, SEQ ID NOs: 29 and 60, or SEQ ID NOs: 31 and 62.

24. A pharmaceutical composition comprising the hsRNA of claim 19.

25. The pharmaceutical composition of claim 24, wherein the pharmaceutical composition is for preventing or treating an infection, cancer, or for providing an immune response to an antigen.

26. The pharmaceutical composition of claim 25, wherein the infection is a viral or bacterial infection.

27. The pharmaceutical composition of claim 25, wherein the cancer is melanoma, skin cancer, breast cancer, lung cancer, colorectal cancer, pancreas cancer, or a combination thereof.

28. The pharmaceutical composition of claim 25, wherein the pharmaceutical composition further comprises an antigen or therapeutic agent.

29. The pharmaceutical composition of claim 28, the therapeutic agent is an anti-cancer drug.

30. A method of treating a disease or disorder comprising administering to a subject in need of such treatment a therapeutically effective amount of the hsRNA of claim 19.

31. The method of claim 30, wherein the disease or disorder is treating an infection, or cancer.

32. The method of claim 31, wherein the infection is a viral or bacterial infection.

33. The method of claim 31, wherein the cancer is melanoma, skin cancer, breast cancer, lung cancer, colorectal cancer, pancreas cancer, or a combination thereof.

34. A method of enhancing an immune response to an antigen in a subject comprising administering to a subject in need of such treatment a therapeutically effective amount of the hsRNA of claim 19.
